(19) United States
(12) Patent Application Publication
(10) Pub. No.: US 2015/0320385 A1
(43) Pub. Date: Nov. 12, 2015

(54) SYSTEMS AND METHODS FOR NONINVASIVE HEALTH MONITORING

(71) Applicant: Eclipse Breast Health Technologies, Inc., Santee, CA (US)

(72) Inventor: Kenneth A. Wright, La Mesa, CA (US)

(73) Assignee: Eclipse Breast Health Technologies, Inc., Santee, CA (US)

(21) Appl. No.: 14/802,771

(22) Filed: Jul. 17, 2015

Related U.S. Application Data
(63) Continuation-in-part of application No. PCT/US2014/012061, filed on Jan. 17, 2014.
(60) Provisional application No. 61/753,785, filed on Jan. 17, 2013.
(60) Provisional application No. 61/753,789, filed on Jan. 17, 2013.
(60) Provisional application No. 62/115,726, filed on Feb. 13, 2015.

(51) Int. Cl. A61B 8/08; A61B 8/00; A61B 5/01; A61B 5/00

(52) U.S. Cl. CPC A61B 8/0825; A61B 5/004; A61B 5/0064; A61B 5/0091; A61B 5/6843; A61B 8/485; A61B 8/565; A61B 5/015; A61B 2560/0431; A61B 2562/0223; A61B 2562/0247

(57) ABSTRACT

Implementations described and claimed herein provide systems and methods for accessible and reliable routine health monitoring and noninvasive detection and early diagnosis of diseases and conditions. In one implementation, a health monitoring device is provided. The health monitoring device includes a light source configured to emit photons into an optical waveguide, which internally reflects the photons. A compliant surface is compressible against the optical waveguide during a scan of tissue. The compression of the compliant surface against the optical waveguide scatters at least one of the photons into the tissue and/or back through the optical waveguide. An imaging array is configured to collect the at least one scattered photon, forming an image representing a hardness of the tissue relative to surrounding tissue.

### CROSS-REFERENCE TO RELATED APPLICATIONS

The present application is a continuation-in-part of and claims priority under 35 U.S.C. §111 to Patent Cooperation Treaty Application No. PCT/US2014/012061, entitled “Systems and Methods for Noninvasive Health Monitoring” and filed on Jan. 17, 2014, which claims priority under 35 U.S.C. §119 to U.S. Provisional Patent Application No. 61/753,789, which was filed Jan. 17, 2013 and entitled “AHST,” and to 35 U.S.C. §119 to U.S. Provisional Patent Application No. 61/753,785, which was filed Jan. 17, 2013 and entitled “Breast Health Examination System.” The present application further claims priority under 35 U.S.C. §119 to U.S. Provisional Patent Application No. 62/115,726, entitled “Systems and Methods for Noninvasive Health Monitoring” and filed on Feb. 13, 2015. Each of the aforementioned applications is hereby incorporated by reference in its entirety into the present application.

### TECHNICAL FIELD

Aspects of the present disclosure relate to routine health monitoring, among other functions, and more particularly to noninvasive detection and early indications or diagnosis of diseases and conditions, such as breast cancer.

### BACKGROUND

For many human diseases and conditions, early diagnosis has a profound effect on survival rate. For example, breast cancer afflicts more than ten percent of American women, with hundreds of thousands of new cases diagnosed per year. Currently, approximately 61 percent of breast cancer incidences are successfully detected at an early stage, and of those cases, the survival rate is approximately 98 percent. Conversely, failure to efficiently diagnose breast cancer may result in the spread of the cancer into nearby tissues and/or distant regions of the body. In such cases, the five year survival rate is as low as approximately 27 percent.

Conventional methods for aiding early detection, even when performed correctly, generally carry a substantial risk of inaccuracy. For example, self-breast exams, while easy to conduct, are often performed by people who are unaware of the signs of a malignant tumor. As such, even a large lump may go undiagnosed for some time.

Mammograms are often utilized as a supplement to self-breast exams, providing a visualization of any malignancies. However, mammograms are generated using high-energy radiation, which can be dangerous, and in rare cases, lead to the development of cancer. Additionally, mammograms are highly prone to human error and/or inconclusive. Specifically, mammograms show only the shadow of a tumor and fail to reach important areas like lymphatic system near the upper arm/chest region. Thus, detection relies heavily on the interpretation of such shadows by a trained physician. Based on this reliance, physicians have overlooked up to 29 percent of tumors that would have been detected by their peers.

While nuclear magnetic resonance imaging (MRI) techniques may reveal intricate details of the size and shape of a tumor, the resolution is still too low to detect relatively smaller tumors, and such techniques are generally complicated, time-intensive, and expensive, further reducing effectiveness in aiding early detection. Exams utilizing conventional optical methods generally involve the injection of a fluorescent stain or other foreign compound, which often deters people from regularly obtaining such exams. Additionally, such optical techniques may be prone to interference from the size and shape of the patient's body and/or the fluorescence of surrounding tissue, thereby scrambling the processing of optical signals. Addressing the scrambling requires complex analysis, which may introduce errors, including the production of false positives. Other modern techniques, for example involving the systemic distribution of a chemical marker or the use of biomarkers, similarly require the patient to receive an injection. These techniques are often performed over two separate appointments: one to perform the injection; and one to perform a test after a certain period of time has elapsed since the injection.

The primary conduit for early detection of breast cancer and other types of cancer remains regular screening. However, despite an increase in screening, many people still fail to regularly perform or receive exams. Many people lack the knowledge, willpower, access, and/or resources to regularly obtain exams. The side effects and drawbacks of the procedures coupled with the reliability of the results further deter people from obtaining regular exams.

These challenges are exacerbated for patients with or susceptible to other types of cancer, such as lung and bladder cancer. Many of the techniques discussed above are not available to assist in early detection of such cancers.

It is with these observations in mind, among others, that various aspects of the present disclosure were conceived and developed.

### SUMMARY

Implementations described and claimed herein address the foregoing problems, among others, by providing accessible systems and methods for reliable early detection and diagnosis of diseases and conditions. In one implementation, a health monitoring device is provided. The health monitoring device includes a light source configured to emit photons into an optical waveguide, which internally reflects the photons. A compliant surface is compressible against the optical waveguide during a scan of tissue. The compression of the compliant surface against the optical waveguide scatters at least one of the photons into the tissue and/or back through the optical waveguide. An imaging array is configured to collect the at least one scattered photon, forming an image representing a hardness of the tissue relative to surrounding tissue.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 shows an example handheld health monitoring device.

FIGS. 2A-2C illustrate bottom perspective, side, and top views, respectively, of the handheld health monitoring device of FIG. 1.

FIG. 3 shows a side view of the handheld health monitoring device of FIG. 1 in a docking station.

FIG. 4 displays an exploded view of the handheld health monitoring device of FIG. 1.

FIG. 5 illustrates a diagram of an example sensor of a health monitoring device.

FIG. 6 shows a diagram of an example optical sensor of a health monitoring device.

FIG. 7 shows a diagram of an example static or dynamic tactile or wave front sensor of a health monitoring device.

FIGS. 8A-8C show top, side, and bottom views, respectively, of an example health monitoring device with a rolling sensor head.

FIGS. 9A and 9B show bottom and side views of another example health monitoring device with a rolling sensor head in a docking station.

FIGS. 10A-10B illustrate cross sections of front and side views, respectively, of the health monitoring device of FIGS. 9A and 9B with example optical or tactile sensors.

FIG. 100 shows a cross section of a front view of the health monitoring device of FIGS. 9A and 9B with another example of optical or tactile sensors.

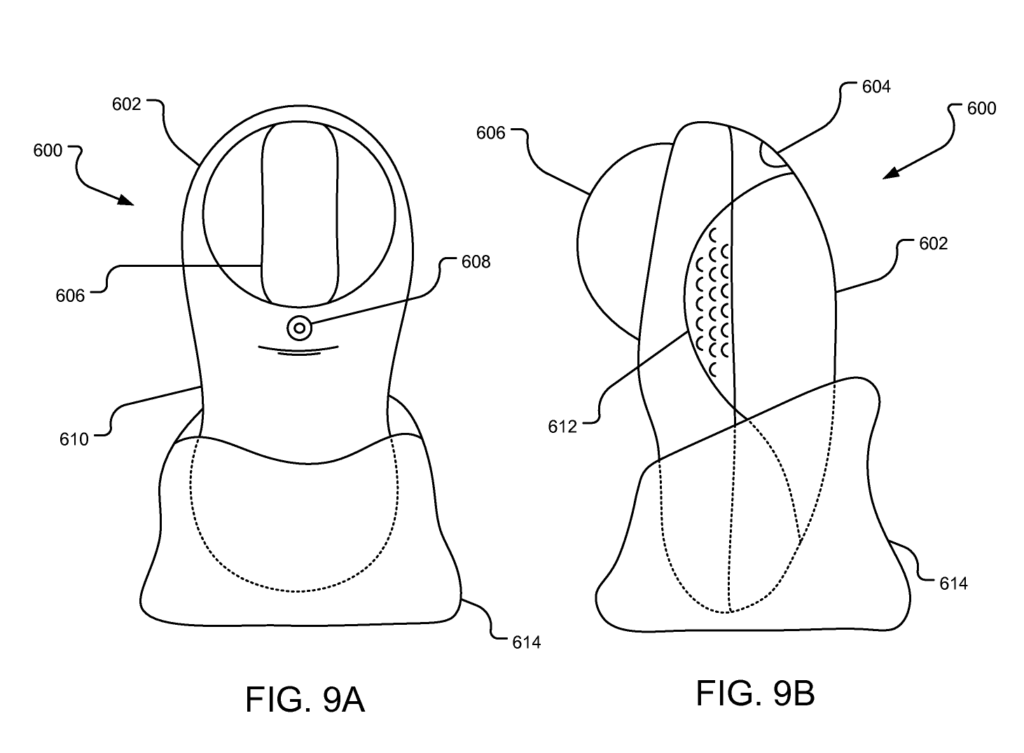

FIGS. 11A-11C illustrate front cross section, side cross section, and front views, respectively of the health monitoring device of FIGS. 9A and 9B with another example of optical or tactile sensors.

FIGS. 12A-14C depict various views of an example health monitoring device with disposable, interchangeable, reversible, or otherwise removable sensor heads.

FIG. 15 shows a side view of an example health monitoring device with a handle.

FIGS. 16A-16C show different views of an example round health monitoring device.

FIGS. 17A-17B illustrate an example finger loop health monitoring device with force activation.

FIGS. 18A-22C show various example health monitoring devices configured to operate using a smartphone or similar user device.

FIGS. 23A-23B illustrate various views of an example health monitoring device for use in spa, beauty, or wellness settings.

FIGS. 24A and 24B illustrate top and side views, respectively, of example coupling material having a guiding pattern for a health monitoring device.

FIG. 25 illustrates an example conductive material to facilitate signal transmission and receipt by a health monitoring device.

FIG. 26 depicts an example system for health monitoring, including a health monitoring device in communication with a user device.

FIG. 27 shows an example user interface generated by a scanning application, the user interface being displayed in a window of a computing device and displaying breast maps for comparison.

FIGS. 28A-28D show various user interfaces illustrating the capture, alignment, and processing of scans.

FIG. 29 illustrates example operations for noninvasive detection and early diagnosis of diseases and conditions.

FIG. 30 is an example health monitoring system, including a health monitoring application running on a computer server, computing device, or other device coupled with a network, for routine health monitoring and noninvasive detection and early diagnosis of diseases and conditions.

FIG. 31 shows an example user interface generated by the health monitoring application application, the user interface being displayed in a window of a computing device and displaying breast maps.

FIG. 32 shows another user interface displaying a comparison of breast maps taken over a time period.

FIG. 33 shows another user interface displaying health monitoring resources, including previous scans.

FIG. 34 illustrates another user interface displaying health monitoring resources.

FIGS. 35A and 35B display top and side views, respectively, of an example clinical health monitoring device.

FIG. 36 shows an example health monitoring device having a mirror interface.

FIGS. 37A and 37B show an example tissue density monitoring device.

FIG. 38 is an example of a computing system that may implement various systems and methods discussed herein.

### DETAILED DESCRIPTION

Aspects of the present disclosure involve apparatuses, systems, and methods for accessible and reliable routine health monitoring and noninvasive detection and early indications or diagnosis of diseases and conditions. The apparatuses, systems, and methods facilitate the performance of an exam, such as a breast exam, in various environments, including a patient's home, a hospital, a doctor's office, a clinical setting, a mobile setting, a fitness center, an alternative medicine center, wellness center, retail outlet (e.g., a drugstore), spa, or the like. Further, apparatuses, systems, and methods compare results from current exams of patient tissue to previous results to determine any changes in the tissue using a baseline reading of the tissue. Identification of any changes generates a communication to prompt the patient or healthcare provider to seek additional medical advice, testing, and/or diagnostics regarding the patient tissue.

In one aspect, a health monitoring system involving one or more a health monitoring device is provided, including one or more sensors. The sensors may include, without limitation, an optical sensor, a static tactile sensor, a dynamic tactile sensor, a red-green-blue (RGB) sensor, a Near Infrared (NIR) sensor, a thermal imaging sensor, a passive sensor, a skin chemical sensor, a waste chemical sensor, a microphone, a depth sensor, a stereoscopic sensor, a scanned laser sensor, an ultrasound sensor, a multiple wave sensor, a force sensor, and the like.

The health monitoring system facilitates access to reliable early detection of human diseases and conditions, such as breast cancer, through direct detection and the monitoring of physical and/or chemical changes over time. Performance of exams is simple, affordable, understandable, and efficient. During an exam, health information for a patient is obtained through the collection and processing of data collected by the one or more sensors. The health information may be processed, for example, using: the health monitoring device; a computing device; a remote computer server or device at a centralized location, such as a doctor's office, medical laboratory, or the like; and/or using a secure cloud-based application running on a computer server and accessible using a user device. The health information may be used to identify the possible presence of a disease or condition and to monitor any changes. Diagnostic results and corresponding information are delivered to the patient in an understandable manner, reducing the reliance on human interpretation of data. As such, exams may be regularly performed and analyzed by a layperson, an assistant, and/or a trained professional.

In one particular aspect, the health monitoring device is a pressure point sensing device that may be used as an adjunct to traditional Breast Self-Examinations (BSE). The device locates and documents features found during a routine BSE by collecting digital image data for reference. During an exam, a user, such as the patient, scans the device over a breast in a systematic pattern. The device provides a digital pressure-based map of the scanned breast that may be stored, analyzed, or discussed with a health care provider. More specifically, in one implementation, the device includes a light source, an optical waveguide, and a compliant surface or other opaque material. The light source emits light into the optical waveguide, which internally reflects the light. During an exam, the pressure of the breast tissue against the compliant surface compresses the compliant surface against the optical waveguide. The harder the tissue (e.g., in the presence of a hard lump or lesion) the more the complaint surface compresses the optical waveguide. As the compliant surface is compressed, the light reflected in the optical waveguide is back-scattered to a sensor, such as a camera, producing an image capturing the relative hardness and softness of the scanned tissue. Therefore, relatively hard tissue, possibly indicative of a tumor, will appear in the image captured by the camera. Regular exams will reveal any physical changes of such hard tissue over time.

In some implementations, in addition or alternative to passive or reactive transmissions (e.g., pressure, palpation, tactile, thermography, etc.), the health monitoring device is configured to generate and read multiple wave fronts to provide active dynamic-variable transmissions. Such wave fronts may include, without limitation, percussive (e.g., mechanical pulses approximately 1-100 Hz), pulse modulation (e.g., vibratory), sonic (e.g., 100-10000 Hz), photonic (NIR, full spectrum variable), electronic, thermal (e.g., with cold challenge), mechanic, and the like. The various multiple wave fronts provide a noninvasive signal that may be read back to detect different tissue densities, pressures, patterns, changes, and/or the like. One or more sensors of the health monitoring device configured to generate and read the various passive, reactive, and/or active dynamic-variable transmissions may be included in a sensor head, which may be actuated in various manners. The actuation of the sensor head may involve, without limitation, rolling, gliding, pressing, rocking, and other dynamic or static actuations. The sensor head may be optionally removable or interchangeable.

Further, in some implementations, the health monitoring device includes one or more target enhancements to facilitate signal transmission and receipt. Such target enhancements may include, without limitation, touch-down pads with various geometries, textures, and/or materials; mechanical enhancements, such as waveguide and/or sonic enhancements; conductive materials, such as gels and/or pressure plates; compression enhancements, including movement dynamics orientation; placement enhancements, for example, involving gravity, magnetics, and/or electro-mechanical aspects; automation, including robotics, stabilization, and/or vibration; and/or thermal enhancements, including photonic and/or electronic.

The various apparatuses, systems, and methods disclosed herein provide for accessible and reliable routine health monitoring and noninvasive detection and early diagnosis of diseases and conditions. Some of the example implementations discussed herein reference the detection of cancer in humans, and more particularly breast cancer. However, it will be appreciated by those skilled in the art that the presently disclosed technology is applicable to other human and non-human diseases and conditions.

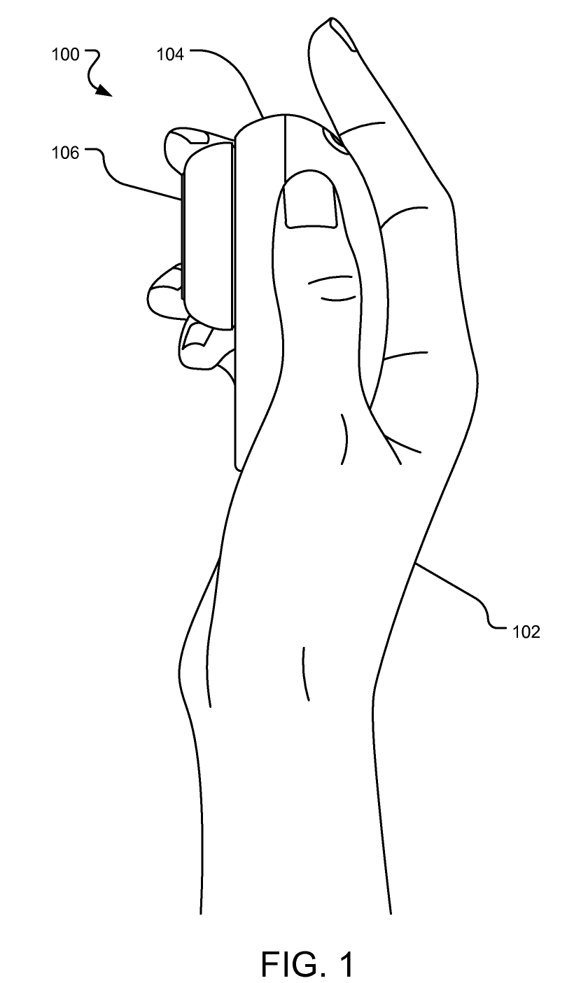

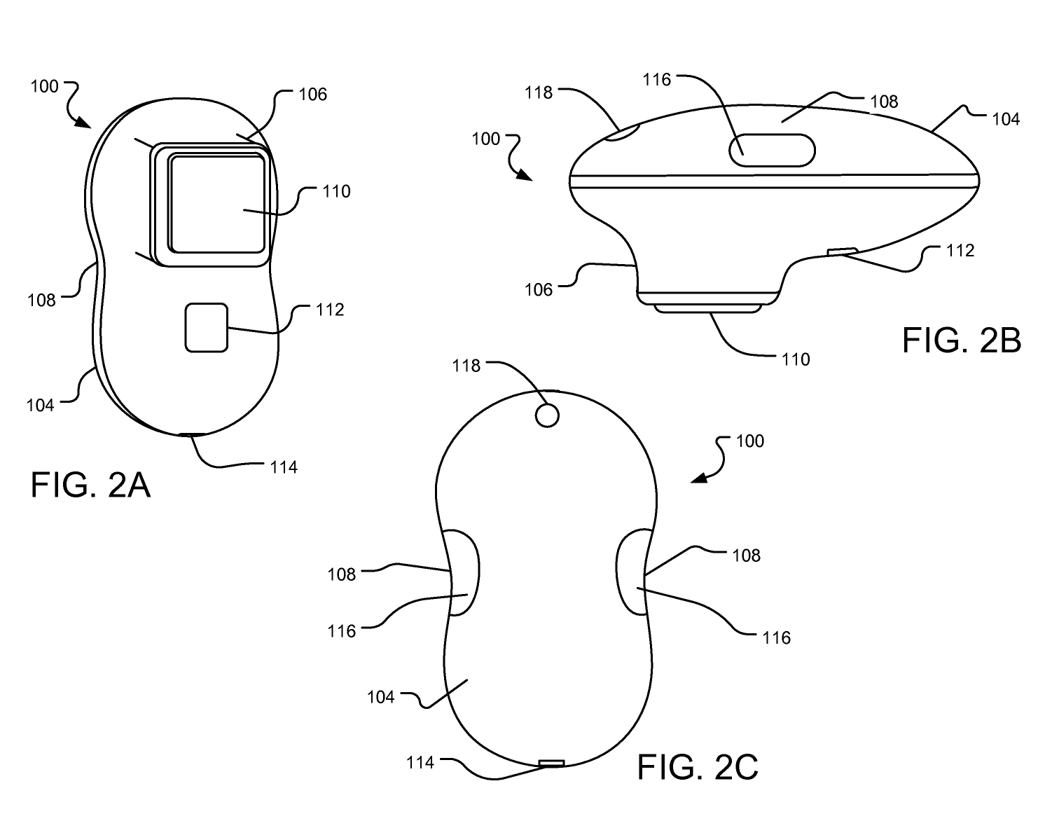

For a detailed description of an example handheld health monitoring device 100, reference is made to FIGS. 1 to 2C. As can be understood from FIG. 1, the device 100 is sized and shaped to comfortably fit in a hand 102 of a user. In one implementation, the device 100 includes a body 104 and a protruding portion 106 extending outwardly from the body 104.

The body 104 may have various surface features, angles, and/or contours to facilitate use and enhance comfort. For example, as shown in FIGS. 2A-2C, the body 104 may be shaped like a computer mouse having surface contours 108 matching the shape of the hand 102. The protruding portion 106 may be a variety of shapes, including, but not limited to, spherical, cubical, conical, elliptical, angular, contoured, convex, or the like. The protruding portion 106 may be adapted to move relative to the body 104 during an exam as the device is moved along the surface of the scanned tissue. For example, the protruding portion 106 may have a rounded shape that rotates during an exam.

In one implementation, the body 104 and/or the protruding portion 106 house one or more sensors. The sensors may include, without limitation, an optical sensor, a static tactile sensor, a dynamic tactile sensor, an RGB sensor, a NIR sensor, a thermal imaging sensor, a passive sensor, a skin chemical sensor, a waste chemical sensor, a microphone, a depth sensor, a stereoscopic sensor, a scanned laser sensor, an ultrasound sensor, a multiple wave sensor, a force sensor, and the like. For example, the body 104 may include a camera 112 or motion sensor disposed near the protruding portion 106 to detect tissue surface features, translation along the surface of the tissue, and the orientation of the device 100 relative to the tissue.

As can be understood from FIGS. 2A-2C, to operate the device 100 during an exam, a surface 110 of the protruding portion 106 is pressed against the target tissue (e.g., breast tissue), and the device 100 is moved systematically over the target tissue, for example, along a guide pattern. In one implementation, the surface 110 comprises a material that maintains a soft or pleasant sensation against the skin, including, without limitation, one or more of latex, vinyl, polypropylene, silicone, or other plastics. The surface 100 may contain a surface lubricant or lotion to facilitate smooth motion against the skin. The body 104 may include one or more grips 116 comprising rubberized or frictional pads to aid in the retention of the device 100 in the hand 102.

In one implementation, to enhance the clarity of the exam results, the device 100 may be rocked or gyrated, by the user or automatically, during the scan of the target tissue. The surface 110 may have chamfered or rounded edges to facilitate such motion. As the device 100 is moved, the sensors collect data corresponding to the target tissue. The data collected by the sensors is processed and analyzed by the device 100 and/or one or more other components of a health monitoring system. As shown in FIGS. 2A-2C, in one implementation, the device 100 includes a USB port 114 for connecting to a user device via a USB cable. In another implementation, the device 100 transmits data for storage, processing, analysis, or the like over a other wired, wireless (e.g., Wi-Fi, Bluetooth, etc.), or network connection (e.g., Wi-Fi, CDMA, CDMA2000, WCDMA, LTE, etc.).

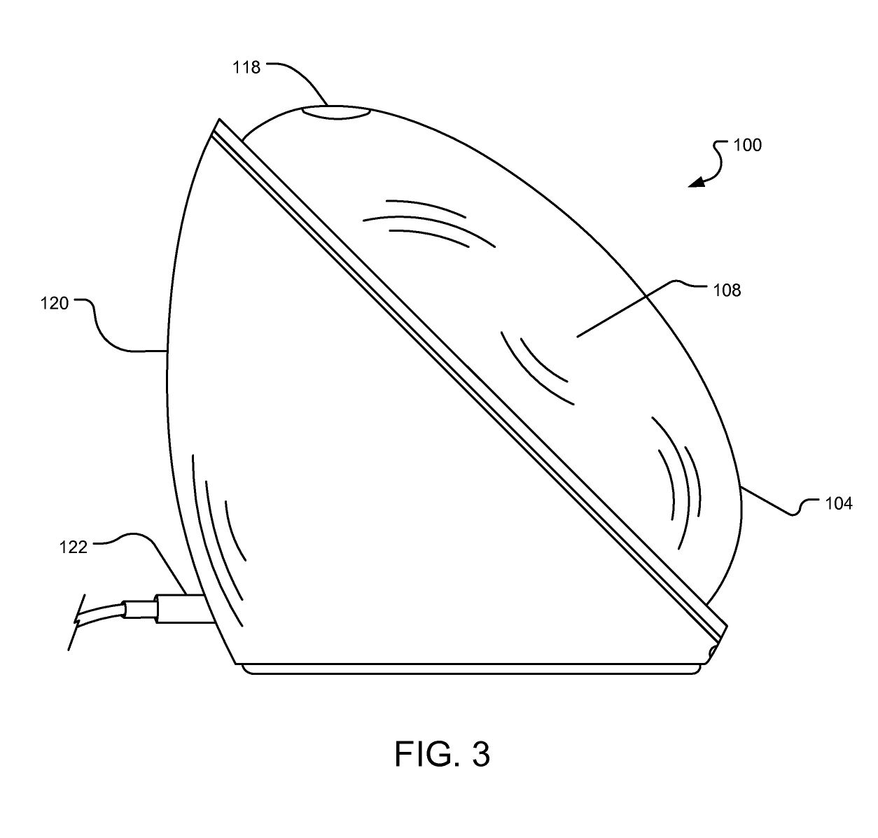

For a detailed description of a docking station 120, reference is made to FIG. 3, which shows a side view of the device 100 resting in the docking station 120. In one implementation, the docking station 120 charges the device 100 through power drawn from a power supply, which may include, without limitation, an electrical outlet, a battery supply, parasitic power from a computing device (e.g., via a USB connection), collected solar power, or the like. For example, as shown in FIG. 3, the docking station 120 may include a cable 122 for connecting to an electrical outlet, Universal Serial Bus (USB) port, or other power source to draw power. In one implementation, the docking station 120 is configured to collect data from the device 100 and transmit the data via the cable 120 or wirelessly to a computing device and/or over a network.

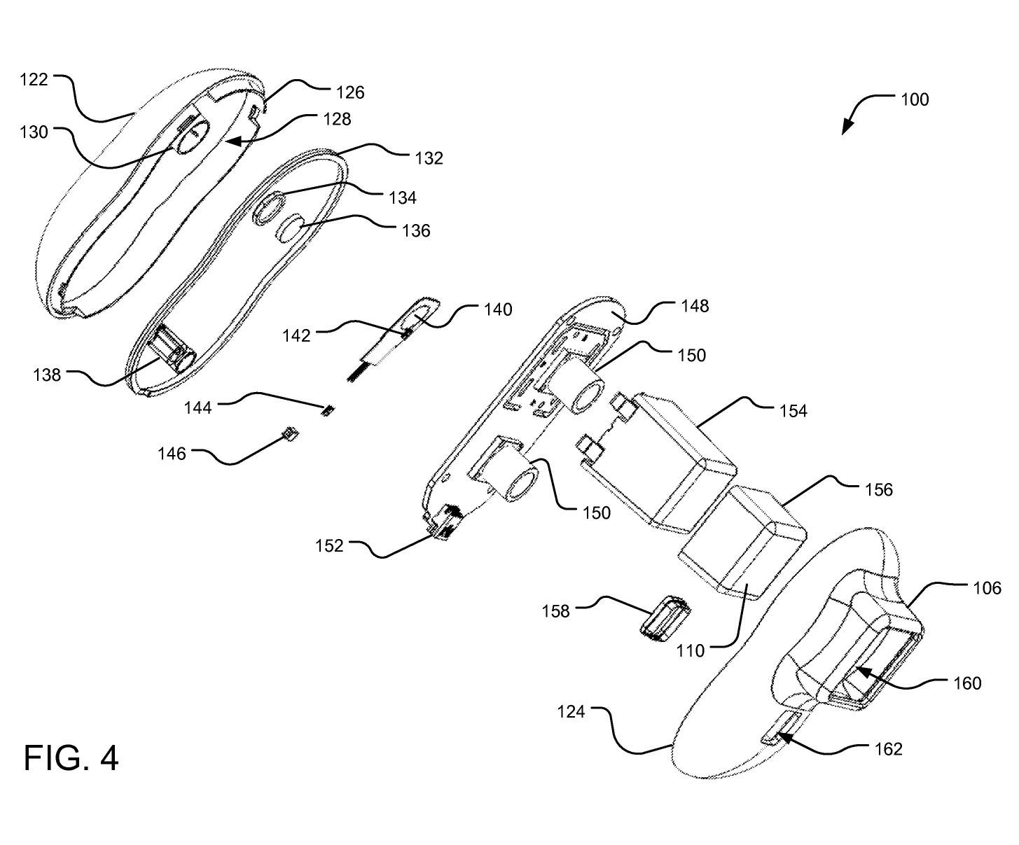

Referring to FIG. 4, an exploded view of the device 100 is shown. In one implementation, the body 104 of the device 100 includes a first cover 122 and a second cover 124. The first cover 122 includes male engaging members 126 to engage corresponding female members of the second cover 124 to enclose the body 104 to form an interior housing 128. In one implementation, the covers 122 and 124 may be removed to disassemble the device 100 to facilitate replacement, disposal, cleaning, and/or upgrade of the components of the device 100.

The interior housing 128 contains interior components of the device 100. In one implementation, the first cover 122 includes a protruding section 130 for positioning a belt 132. The protruding section 130 is disposed relative to a cushion support 134 of the belt 132. A cushion 136 is positioned between the cushion support and a sensor 140. In one implementation, the sensor 140 is a pressure sensor for use in conjunction with a corresponding image capture button on the first cover 122 to capture images based on the user's input. In this instance, the cushion 136 provides controlled pressure to the sensor 140 from the image capture button. In one implementation, the belt 132 further includes a light pipe 138 positioned relative to a light source 146, such as a light emitting diode (LED). The light source 146 may provide visual status indications to the user.

The device 100 includes one or more additional sensors 142, 144 to collect health data. The sensors 142, 144 may include one or more of an optical sensor, a static tactile sensor, a dynamic tactile sensor, a red-green-blue (RGB) sensor, a thermal imaging sensor, a passive sensor, a skin chemical sensor, a waste chemical sensor, a microphone, a depth sensor, a stereoscopic sensor, a scanned laser sensor, an ultrasound sensor, a multiple wave sensor, and the like.

Where the sensors 142, 144 are used as part of an optical sensor, the device 100 emits and collects light in the visible and/or near-infrared wavelengths. The device 100 transmits light, either continuously or with short pulses, into and through target tissue to image the structure of the tissue, including interior tissue well below the skin. Examples of information that may be obtained by an optical sensor in one or more wavelength bands includes, without limitation: transmission, reflectance, absorbance, elastic scattering, spectral modulation, fluorescence, auto-fluorescence, phosphorescence, modulation of polarization, Raman scattering, photon Doppler shifting, path speed (index) modulation or retardation, beam focusing or defocusing, Schlieren interferometry, and the like. The sensors 142, 144 may further include a trackball or optical sensor and/or a gyroscopic, magnetic, or other positioning sensor to collect and log the location and orientation of the device 100 relative to the tissue surface. The location and orientation information may be used to process and register (e.g., stitch together) the images collected using the sensors 142, 144, as described herein.

The sensors 142, 144 can be utilized as part of a static tactile sensor, which reads tactile information from the surface of the target tissue. Malignant tumors possess various physical properties that are measurably different from normal tissue, including, for example: decreased elasticity; increased hardness; changes in bulk or shear modulus or other stress-strain quantity; bulging or inflammation; electric properties, including capacitance and inductance, electric impedance, electric potential, or electro-mechanical properties; heat or thermal emission or conduction; plasticity; acoustic or ultrasonic properties; and pressure wave deflection or refraction. As described herein, based on such properties, the static tactile sensor captures images of regions including malignant tumors with a camera.

Similarly, where the sensors 142, 144 are used as part of a dynamic tactile sensor, the device 100 includes a sonic or ultrasonic transducer and receiver for imaging deep tissue. In one implementation, a signal is channeled into the tissue by a device that rests on the surface of the tissue, inducing vibrations in the tissue. The modulations of the signal may be captured by the sensors 142, 144. In this case, ultrasonic imaging, palpitating the tissue (by hand or with a probing device), and scanning the sensors 142, 144 over the surface of the tissue, returns a map of information about the elasticity of the tissue. Because lower elasticity is a strong indication of malignancy of tumors, any potentially malignant tumors present in the tissue may be flagged.

In one implementation, the sensors 142, 144 include a thermal imaging sensor, which records images in mid-wave infrared wavelengths. To increase the quality of the data captured by the thermal imaging sensor, a change in the temperature of the target tissue is induced, for example, through exercise or the application of a controlled cooling or heating device to the target tissue. The thermal imaging sensor tracks the propagation of heat across the surface of the tissue. Because the surface temperature of the tissue is affected by the propagation of heat from points inside the body, any tumors may accelerate or delay the propagation of heat to some points on the surface tissue. Tracking these points and comparing information from previous exams may provide an indication of the presence of a tumor.

The sensors 142, 144 may include one or more passive sensors, which may provide additional information about a patient's overall health. For example, the passive sensors may be used to monitor heart rate, skin conditions, body mass index, blood oxygenation, body temperature, body chemical outgassing, and/or other bodily functions or conditions.

During the course of daily activity, the body emits chemicals through the skin, some of which may be particular biomarkers for cancer, especially volatile chemicals. The dynamics of volatiles inside the body and skin is relatively well understood, and saturation takes place typically on a timescale of hours. One biomarker that is a byproduct of malignant tumors is formaldehyde, which is difficult to detect because it decays and disperses under environmental conditions. Accordingly, the sensors 142, 144 may include a skin chemical sensor for detecting the presence of volatiles indicative of malignant tumors.

In one implementation, the skin chemical sensor is used in conjunction with a garment worn by a patient in different conditions, such as while asleep, bathing, exercising, or the like. The garment is made of or contains a substance which absorbs chemicals from the body during wearing. For example, the garment may include patches positioned near target tissue (e.g., the breasts); the patches including such a substance. The garment collects formaldehyde and quickly transforms it into a chemical with a longer lifetime fixed inside the material of the garment. The skin chemical sensor identifies the concentration of the fixed chemical, which provides an initial concentration of formaldehyde. The garment may be removed for remote analysis using a skin chemical sensor. A probable location of any malignant tumors may be identified by analyzing the portion of the garment containing higher concentrations of the fixed chemical.

In another implementation, the skin chemical sensor performs a gas chromatography/mass spectrometry (GC/MS). For example, the garment or portion of the garment is embedded in a vacuum system, possibly after being dissolved in a solvent solution to re-release the volatile chemicals into gaseous form. A sensitive chromatography system analyses the components of the gas to determine whether a malignant tumor may be present. Alternatively or additionally, the garment or portion of garment may be placed in front of dogs or other animals trained to recognize the signature scent of breast cancer tumors or other biomarker signatures. If the garment is identified the animals a threshold amount of times, the garment is flagged as potentially corresponding to a malignant tumor. The analysis may be performed in sections to identify the portion of the garment containing the strongest emitting area, which likely corresponds to the location of the tumor.

The sensors 142, 144 may be used in conjunction with one or more tools to operate as a waste chemical sensor. Bodily waste generally contains the same biomarkers as skin chemicals, described above. For example, positively identifiable biochemical signatures may be present in urine, blood, and breath. In one implementation, the device 100 may include a balloon into which the patient exhales. The balloon fixes certain chemicals onto its surface over a specific time period, such as several hours. The balloon may be processed by a waste chemical sensor for cancer signatures. It will be appreciated that the device 100 may include a variety of other sensors or components for detecting and analyzing various health functions and conditions.

In one implementation, the device 100 includes a Printed Circuit Board (PCB) having internal electronics, a wired connection port 152 (e.g., the USB port 114) and one or more lens mounts 150. One of the lens mounts 150 is positioned relative to a light pipe cup 154 having a light source assembly and a sensor head 156. The other lens mount 150 is positioned relative to a lens 158. In one implementation, the second cover 124 includes an opening 160 the protruding portion 106 relative to the sensor head 156 and a window 162 in the surface of the second cover 124 relative to the lens 158.

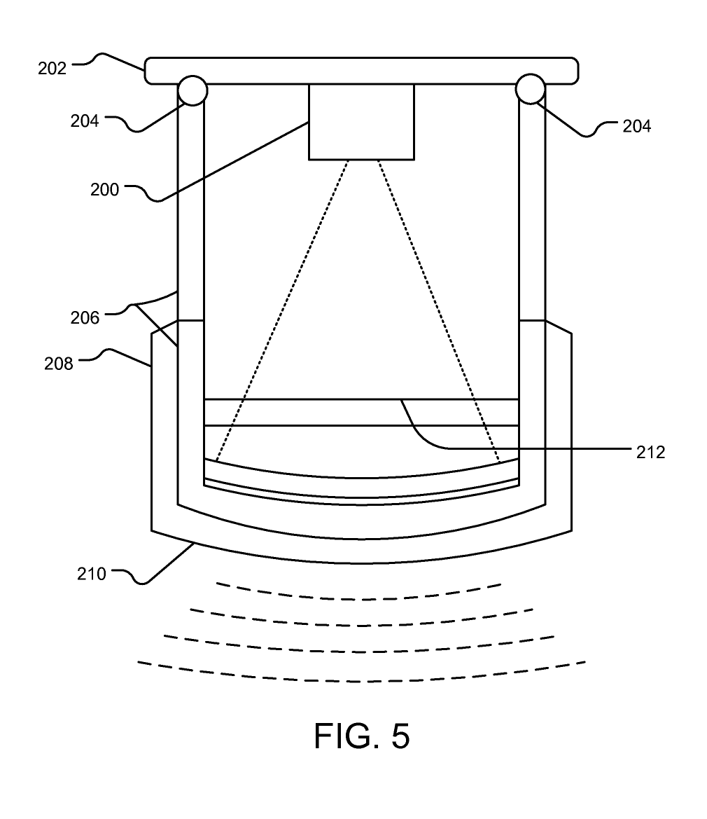

FIG. 5 illustrates a diagram of an example sensor of a health monitoring device. In one implementation, the sensor includes: an imaging array 200, such as Charge-Coupled Device (CCD) camera or other array of optical sensors; a PCB 202; one or more light sources 204, such as LED's, diode lasers, an organic LED, or suitably collimated incandescent light source; an optical waveguide 206; a sensor head 208; a compliant surface 210; and a lens 212.

In one implementation, the compliant surface 210 is pressed against the surface of the target tissue. In another implementation, the sensor transmits a wave front signal and receives a bounce back signal, thereby eliminating or reducing pressure against the target tissue. Light emitted from the light sources 204 is reflected internally in the optical waveguide 206. Due to the physical properties of tumors described above, when the compliant surface 210 is pressed, rolled, or otherwise moved over tissue containing a tumor, lump, or other tissue relatively harder than surrounding tissue, more pressure is exerted onto the compliant surface 210. The increased pressure against the compliant surface 210 compresses the compliant surface 210 against the optical waveguide 206, resulting in frustration of the internal reflection of the light in the optical waveguide 206. Due to natural contours, the amount of frustration is directly proportional to the applied pressure, including at points directly over hardened tissue. A portion of the light escapes from the optical waveguide 206 through the compliant surface 210 into the tissue. The escaped light is scattered directly back through the compliant surface 210 and the optical waveguide 206. The back-scattered light is directed through the lens 212 and captured by the imaging array 200. The captured image resembles a map, in which points receiving more scattered light are those at which the tissue is more tightly pressed against the compliant surface 210, in some cases indicating the presence of an anomaly.

The image map may be processed and analyzed to determine whether the shape, size, and other properties of the hardened tissue indicate it may be malignant cancer. Further, the image map may be compared to image maps obtained from previous exams to determine whether the hardened tissue has grown quickly, possibly indicating the presence of a malignant cancer. In one implementation, a coupling material (e.g., coupling material 500) comprising a material having ribbed, pocked, or otherwise textured features may be placed between the compliant surface 210 and the tissue. Such features or an etched, embedded, or screened on pattern on a surface of the compliant surface 210 may maximize sensitivity of the device in the range of relevant pressures, as well as to facilitate connection with the surface of the tissue with increased traction. Such features or patterns may be tracked optically or using other sensors to track a location and orientation of the device 100.

In one implementation, the device includes a force sensor and display for providing the user with a feedback loop that informs the user of the exerted pressure of the compliant surface 210 against the surface of the tissue in substantially real time, enabling the user to maintain a constant amount of total pressure. Further, the device may include a proximity sensor, permitting the light sources 204 to emit light only when the compliant surface 210 is in close range to tissue, thereby conserving electrical power when an exam is not underway.

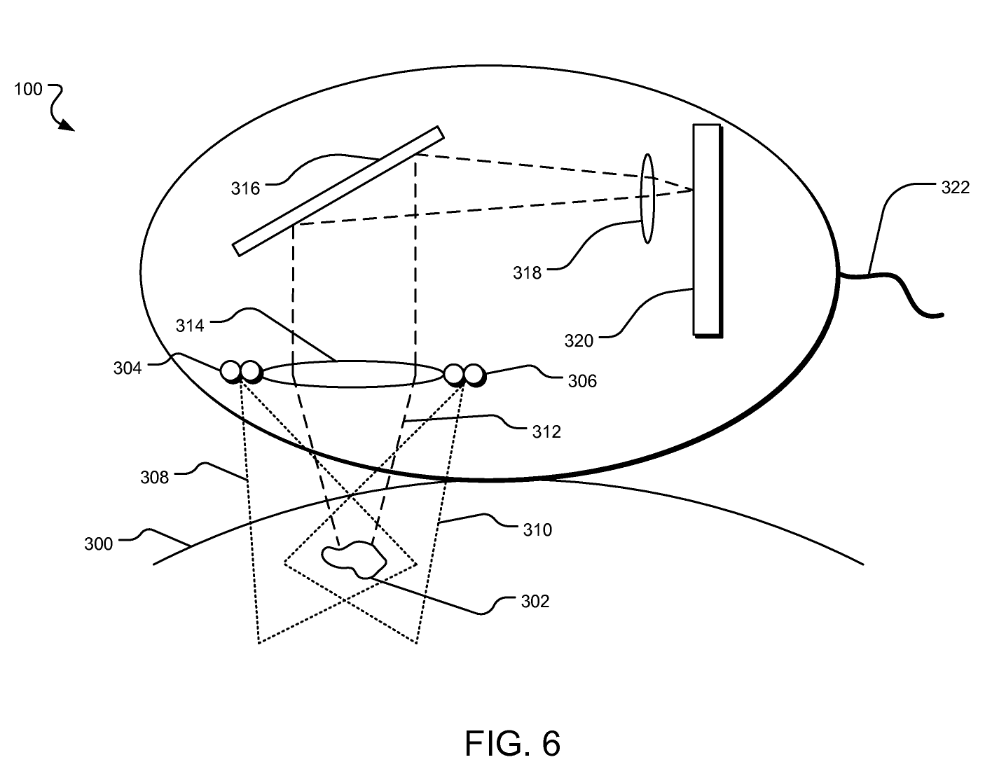

FIG. 6 shows a diagram of an example optical sensor of the device 100. In one implementation, scanning tissue 300 containing a tumor 302 using the device 100 arranged as an optical sensor includes the transmission of light from one or more light sources 304, 306 along an optical path and the collection of such light.

The optical path includes emitted light 308 and 310 from the light sources 304 and 306 respectively into the tissue 300. The light is back-scattered inside the tissue 300 into the device 100, where scattered photons 312 are collected by an element 314. The element 314 directs the photons 312 to a mirror 316, which redirects the photons through collimating optics 318 into a imaging array 320 (e.g., a CCD chip) for collecting the photons as an image. The imaging array 320 exports the received data for processing in locally in the device 100 or remotely via a cable 322 or wirelessly.

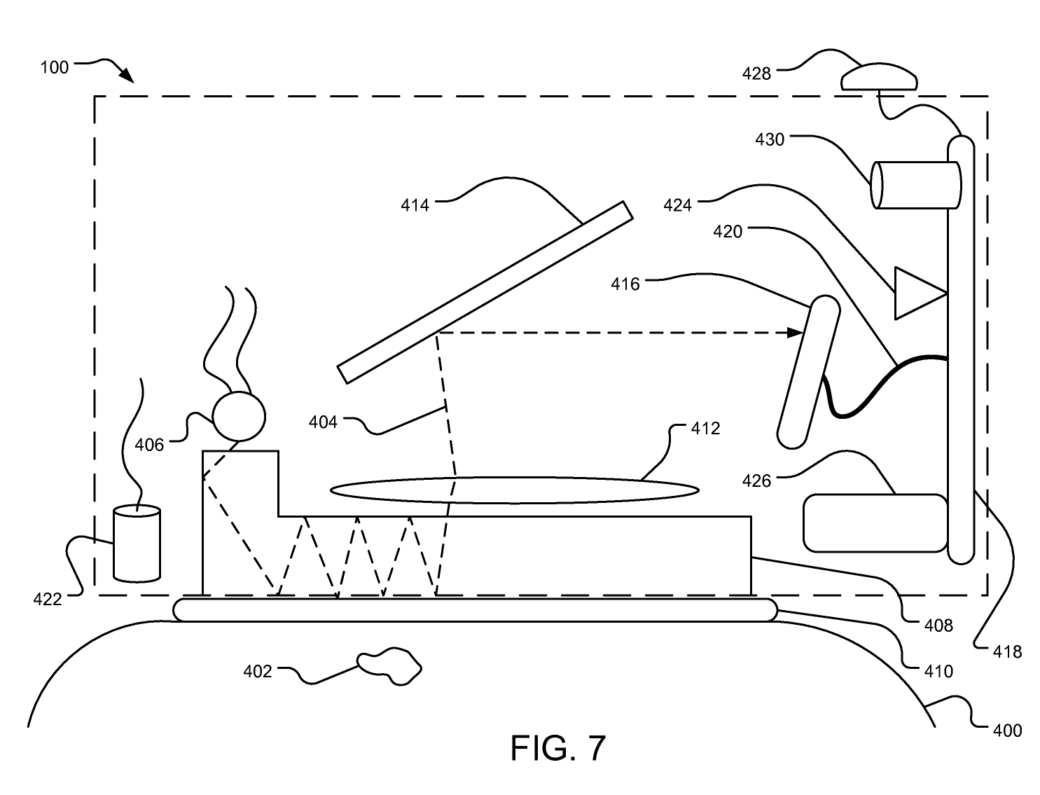

Referring to FIG. 7, a diagram of an example static tactile sensor of the device 100 is shown. As shown in FIG. 7, the device 100 arranged as a static tactile sensor may be used to scan tissue 400 having a relative hard lump 402.

In one implementation, during a scan, the device 100 is pressed, rocked, rolled, or otherwise forcefully contacted to the surface of the tissue 400, as described herein. FIG. 7 illustrates a path 404 of a primary photon during the scanning. A primary photon is a photon that is scattered only in the presence of the hard lump 402 under the surface of the tissue 400. More primary photons are scattered based on the hardness and size of the lump 402. All photons originate at a light source 406 and enter an optical waveguide 408. Within the optical waveguide 408, the photons travel in incoherent directions but are always totally internally reflected at each encounter with a surface of the optical waveguide 408. The photon illustrated in FIG. 7 interacted with the surface of the optical waveguide 408 directly above the lump 402, thereby designating the photon a primary photon.

Due to the enhanced pressure at this point due to the lump 402, a complaint surface 410 is compressed against the optical waveguide 408. The compression provides that the surface of the optical waveguide 408 no longer internally reflects the primary photon due to the relative optical indices of the optical waveguide 408 and the compliant surface 410. As a result, the primary photon travels into the compliant surface 410 where the primary photon is scattered and propagates transversely back through the optical waveguide 408, through a lens 414, such as a Fresnel lens. In one implementation, the primary photon propagates through the lens 414 where it reflects off a mirror 414 and onto an imaging array 416. In another implementation, the primary photon is back-scattered into the device 100 onto the imaging array 416.

The image formed by the captured primary photons may be transferred to a processor 418 or other computing device via a cable 420 or wirelessly. As the device 100 is tracked along the surface of the tissue 400, the image or sequence of images captured is tagged with location and orientation data collected by a sensor 422. The data may be transmitted remotely via a wireless antenna 424 for processing, reconstruction, and analysis. The device 100 may be powered via one or more power sources, such as a battery 426, a wireless charging coil 430, or the like and controlled with an on/off switch 428. It will be appreciated that the device 100 may include addition sensors or components depending on the nature of the scan of the tissue 400. For example, the device 100 may include an embedded RGB camera to capture surface images of the tissue 400 to obtain information regarding surface features, such as moles, dimpling, or other surface skin changes.

In another implementation, the optical waveguide 408 may be replaced with two semi-rigid plates with smooth surfaces and relatively high deformability. Visible, ultraviolet, infrared, or microwave radiation is incident on the plates and interferes with itself from the inner surfaces of each plate, such that the image array 416 images an interferogram showing the deformation of the intra-plate gap. In a location where the hard lump 402 is present, the plates will be sufficiently deformed that a noticeable change or discontinuation of the pattern fringes appears, which may be analyzed to produce a pressure map.

In still another implementation, a plurality of layers is used as a sensing transducer. A first layer proximal to the tissue 400 emits light toward the imaging array 416. A second layer comprises a linear polarizer, and a third layer comprises an optically active material. The orientation of the layers is such that regions under high stress produce proportionally higher modulations of the polarization. A fourth layer distal to the tissue 400 comprises a polarization analyzer. The resultant image thus contains regions of higher or lower intensity and/or dispersion based on the magnitude of the stress induced by pressing the device 100 against the tissue 400. The resultant image may be analyzed to produce a pressure map.

In some implementations, the device 100, for example as described in FIGS. 1-7, includes a sensor head configured to generate and read various transmissions including, without limitation, passive, reactive, and/or multi-active dynamic variable transmissions. The passive or reactive transmissions may include, for example, pressure, palpation, tactile, thermography, and the like. The multi-active dynamic variable transmissions may generally involve multiple wave fronts, including, but not limited to, percussive (e.g., mechanical pulses approximately 1-100 Hz), pulse modulation (e.g., vibratory), sonic (e.g., 100-10000 Hz), photonic (NIR, full spectrum variable), electronic, thermal (e.g., with cold challenge), mechanic, and the like. The various multiple wave fronts provide a noninvasive signal that may be read back to detect different tissue densities, pressures, patterns, changes, and/or the like. The sensor head of the device 100 may be actuated in various manners. The actuation of the sensor head may involve, without limitation, rolling, gliding, pressing, rocking, and other dynamic or static actuations. The sensor head may be optionally removable or interchangeable.

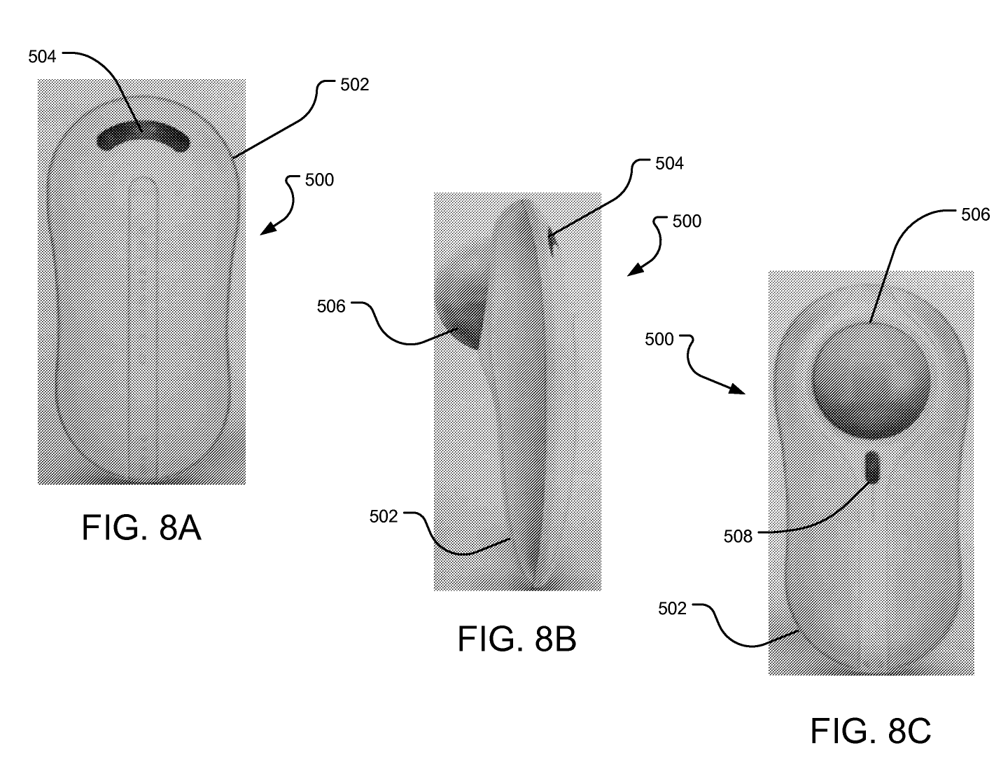

Referring generally to FIGS. 8A-23B, various implementations of health monitoring devices are shown. The health monitoring devices may have similar components and functionality to the health monitoring device 100 described with respect to any of FIGS. 1-7. Moreover, the health monitoring devices of FIGS. 8A-23B may include a sensor head configured to generate and read various transmissions including, without limitation, passive, reactive, and/or multi-active dynamic variable transmissions, as described herein.

Turning first to FIGS. 8A-8C, an example health monitoring device 500 is shown. In one implementation, the device 500 includes a body 502, a user interface 504, a rolling sensor head 506, and an on/off button 508.

The body 502 may be sized and shaped to comfortably fit in a hand of a user. The body 502 may have various surface features, angles, and/or contours to facilitate use and enhance comfort. For example, as shown in FIGS. 8A-8C, the body 504 may be shaped like a computer mouse having surface contours matching the shape of the hand.

The user interface 504 provides feedback to the user and includes one or more options for controlling the operation of the device 500. In one implementation, the user interface 504 includes a visual digital readout and/or other components for providing feedback, such as a speaker to provide audio feedback or light sources to provide other visual feedback. In one implementation, the user interface 504 includes a translucent surface through which the feedback is provided.

In one implementation, the sensor head 506 involves rolling actuation. The sensor head 506 may include or more optical, tactile, or wave front sensors. However, other sensors as described herein are contemplated.

As can be understood from FIGS. 9A-9B, a health monitoring device 600 may be adapted for insertion into a docking station 614. Similar to the various health monitoring devices described herein, the device 600 may include a body 602, a user interface 604, a sensor head 606, an on/off button 608, and grips 612 along contours 610 of the body 602.

The docking station 614 charges the device 600 through power drawn from a power supply, which may include, without limitation, an electrical outlet, a battery supply, parasitic power from a computing device (e.g., via a USB connection), collected solar power, or the like. For example, the docking station 614 may include a cable for connecting to an electrical outlet, Universal Serial Bus (USB) port, or other power source to draw power. In one implementation, the docking station 614 is configured to collect data from the device 600 and transmit the data via the cable or wirelessly to a computing device and/or over a network.

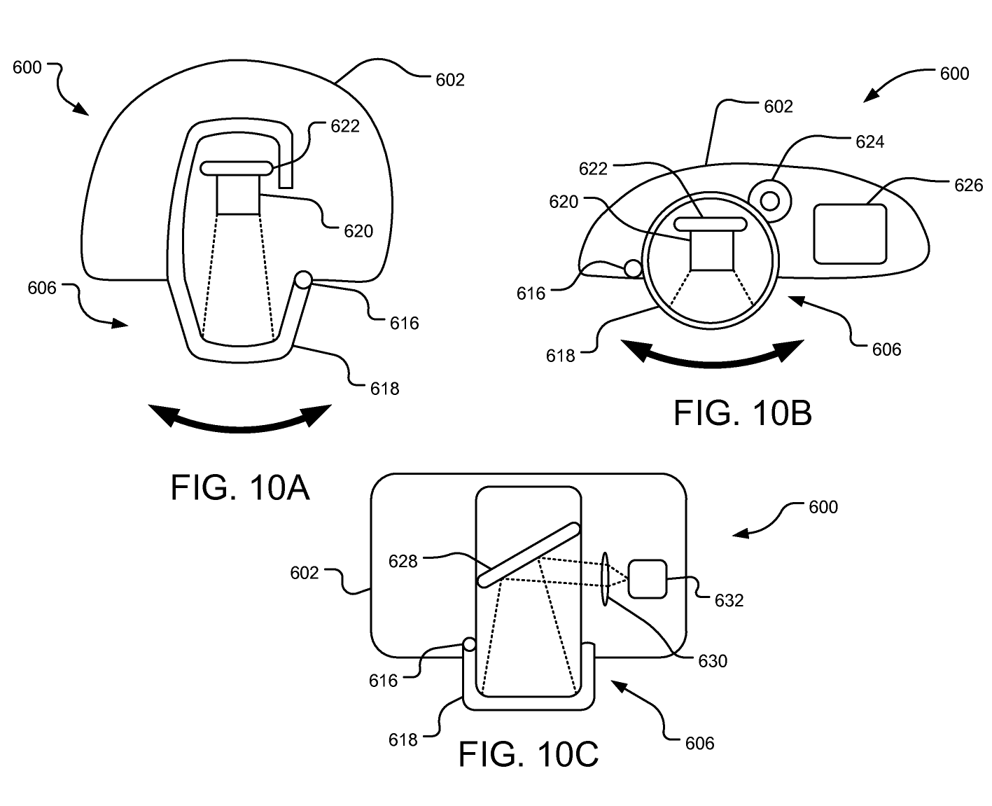

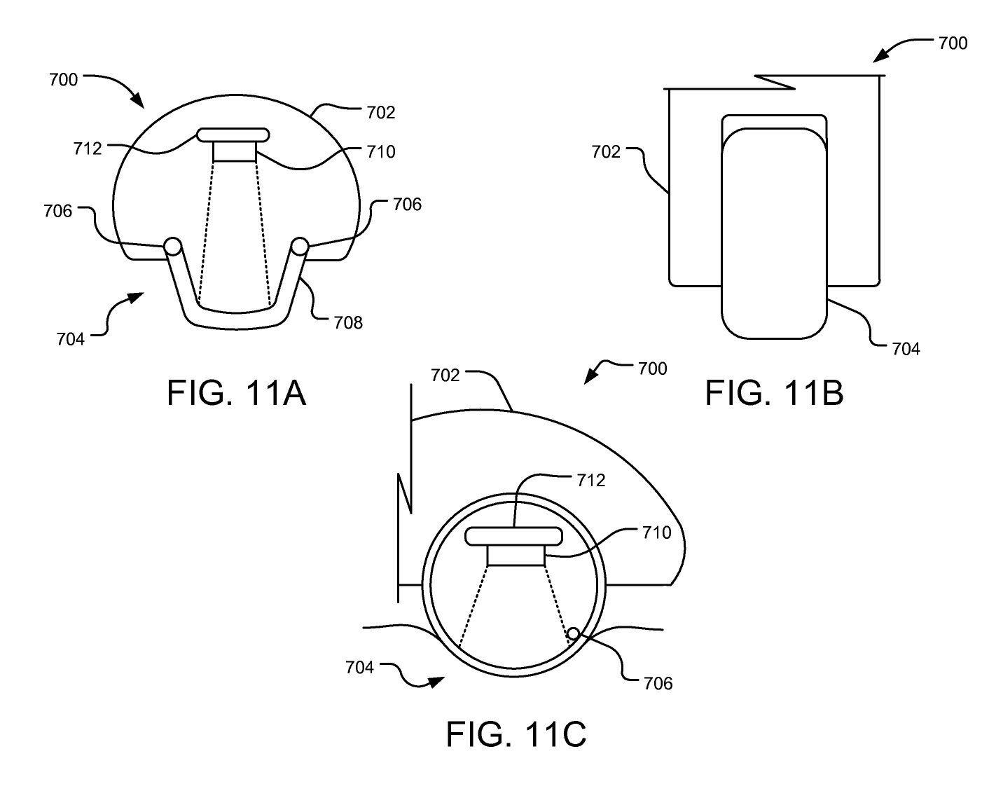

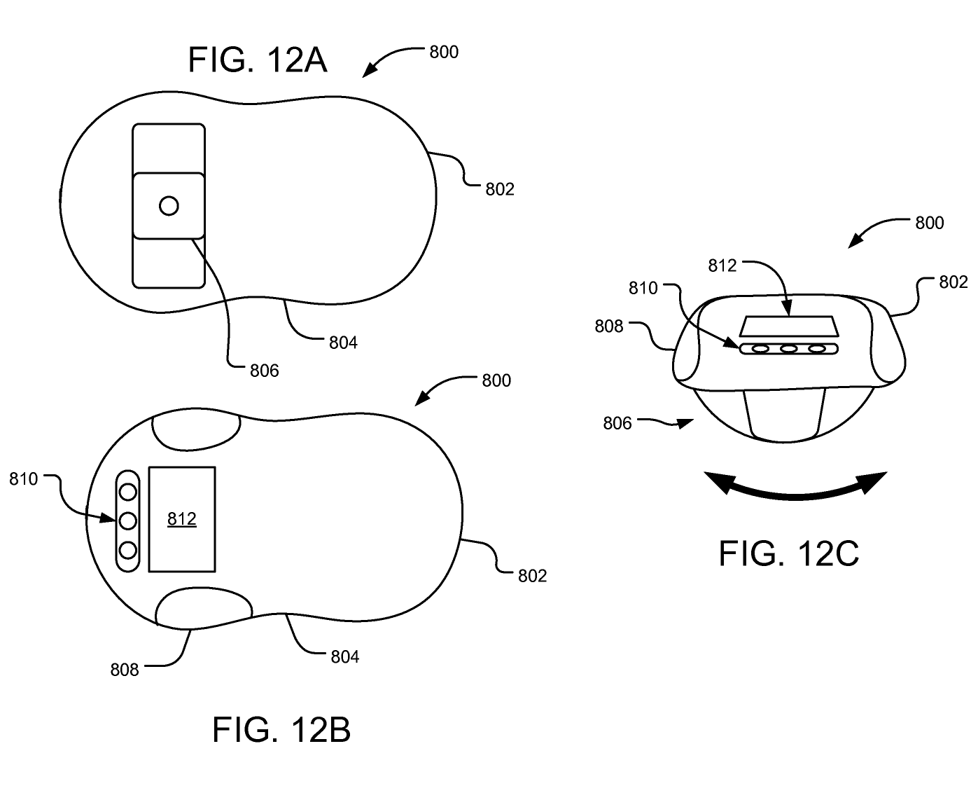

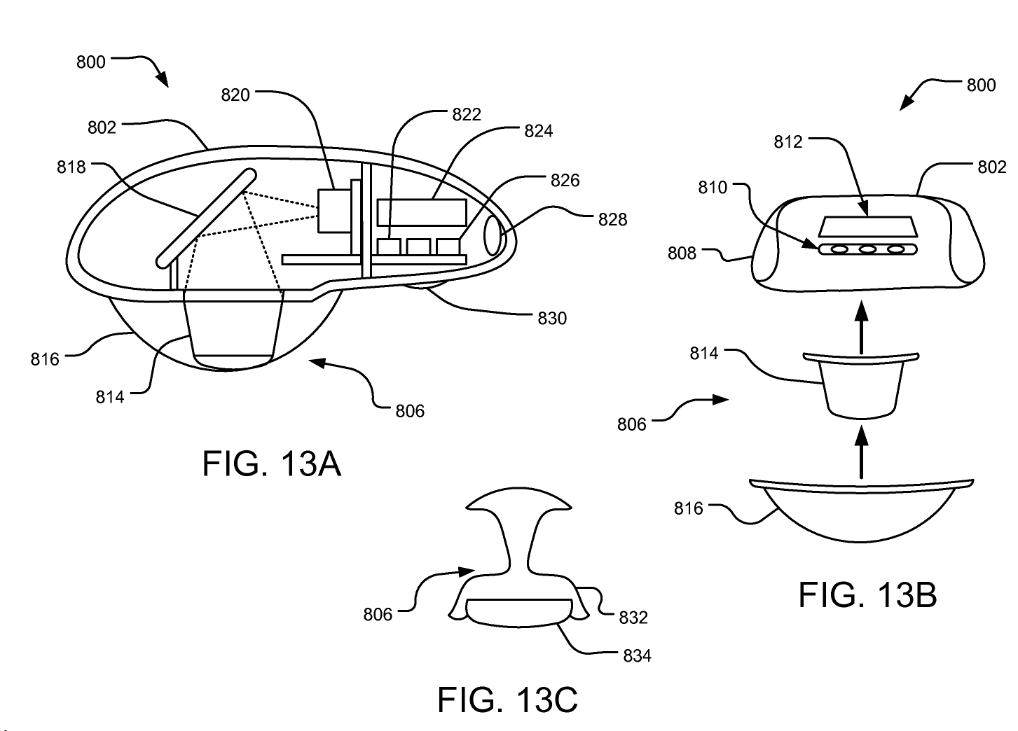

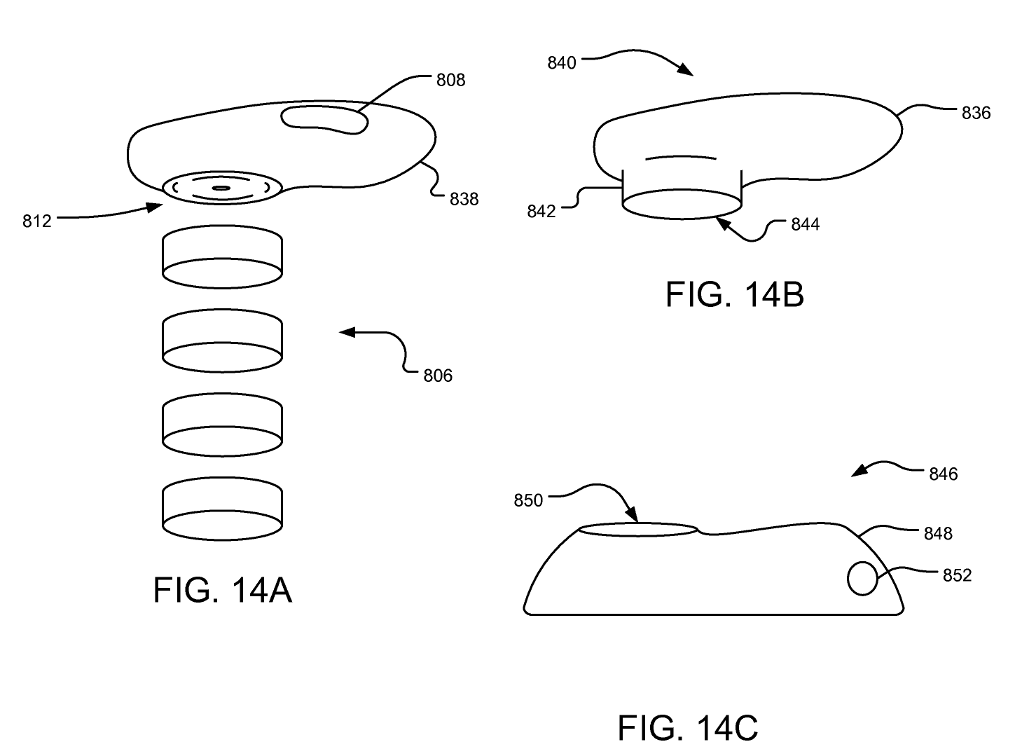

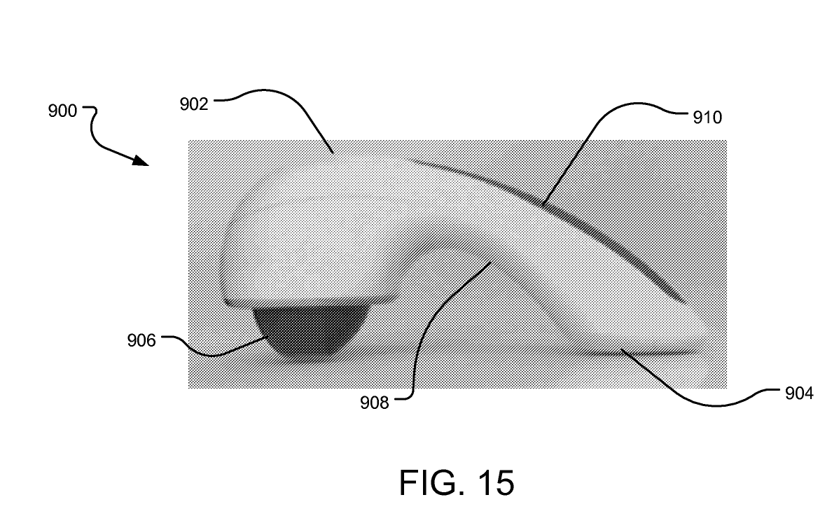

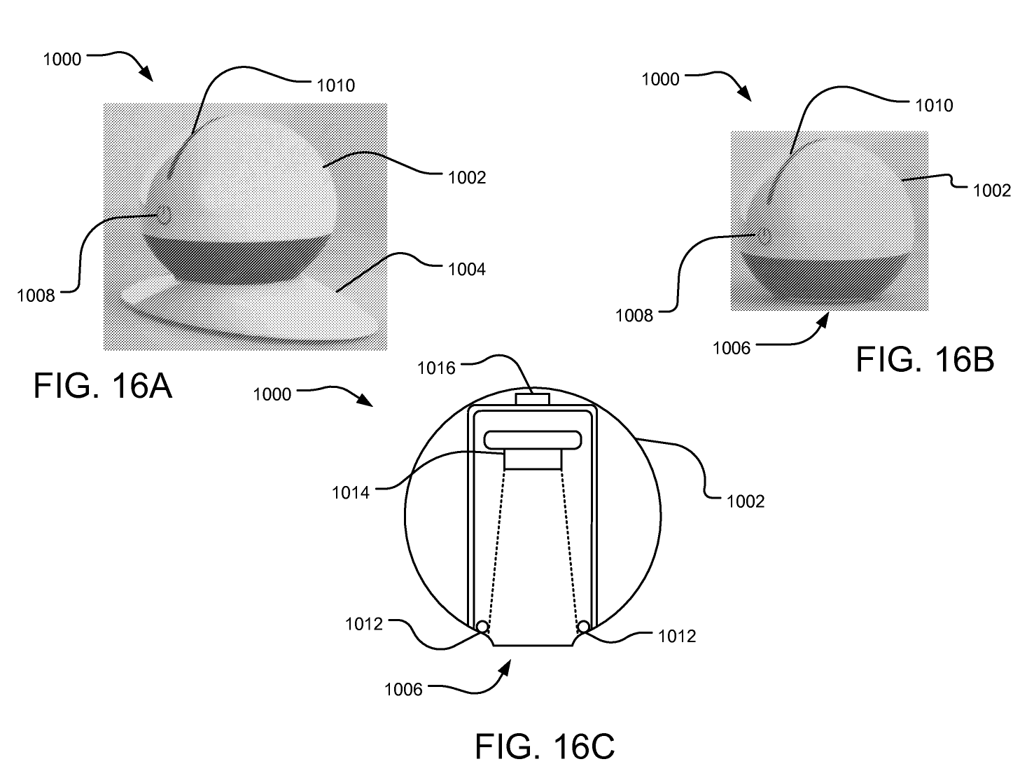

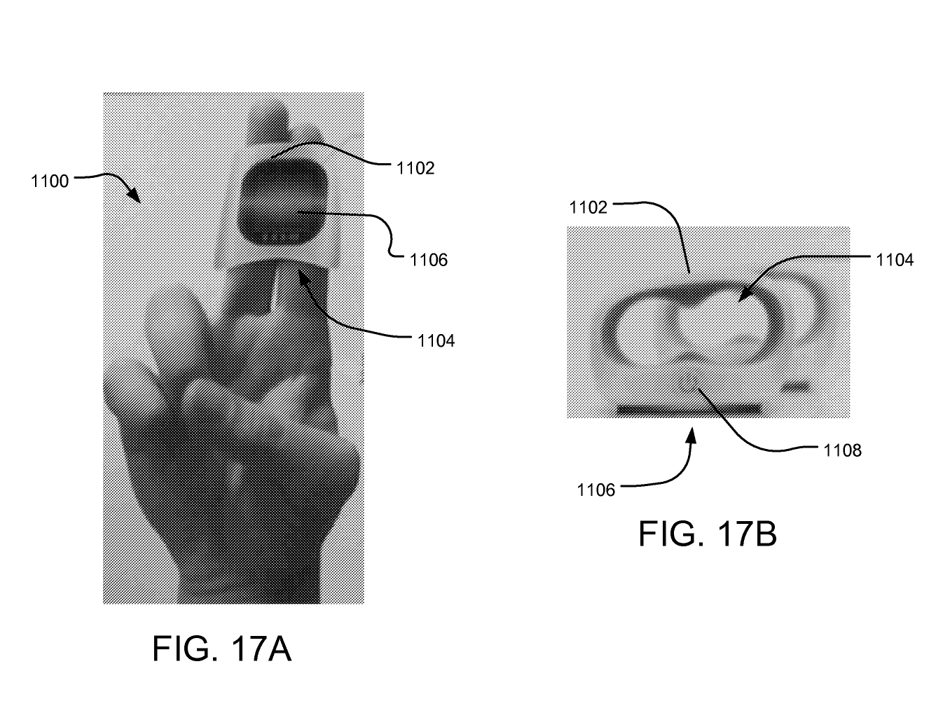

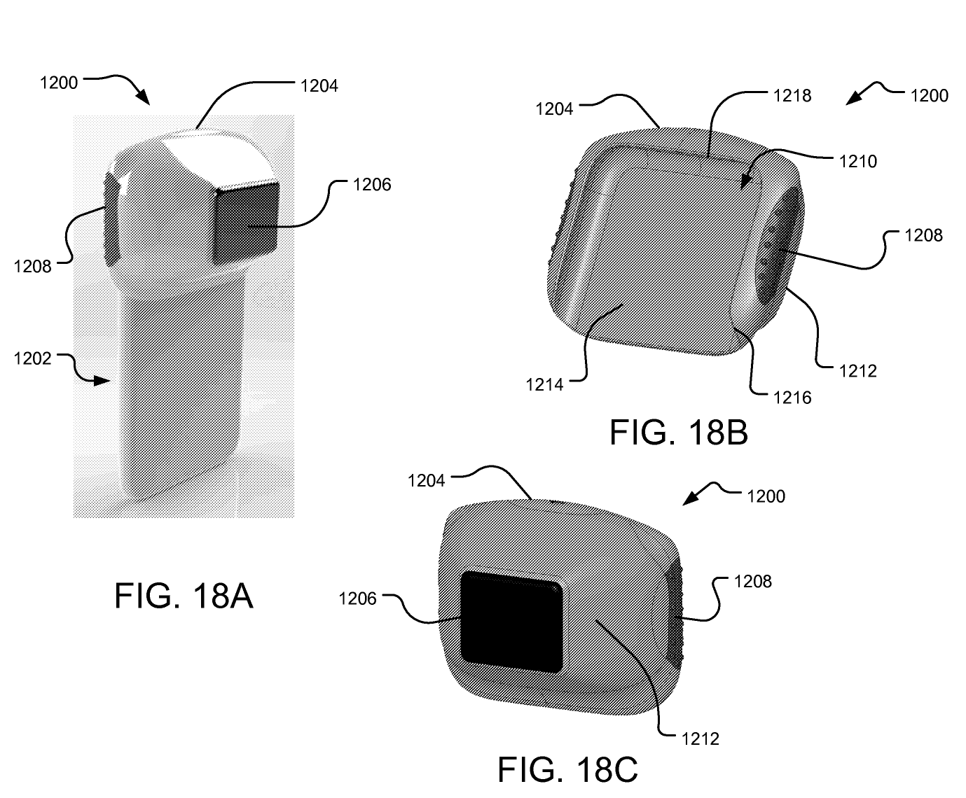

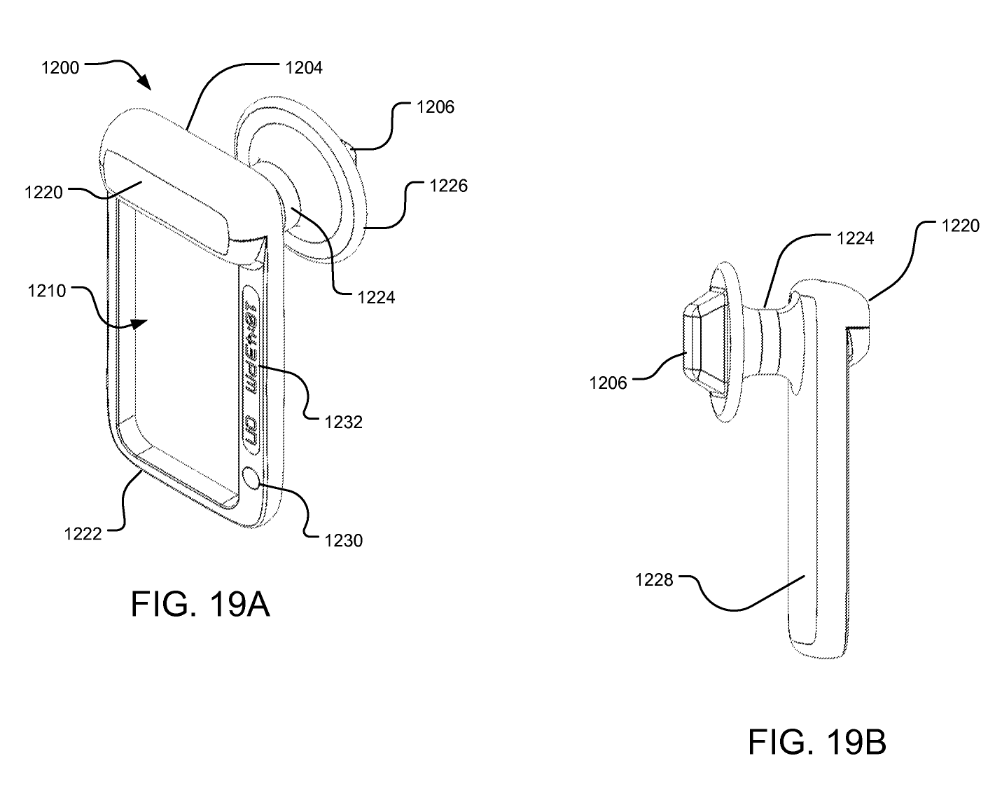

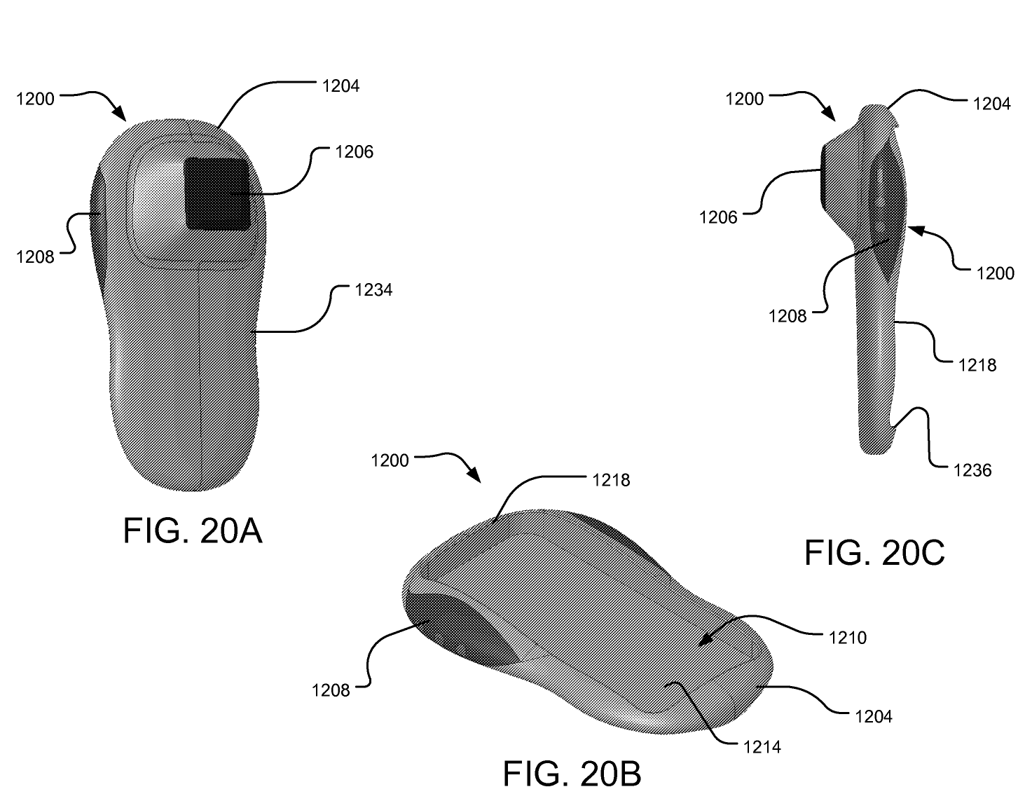

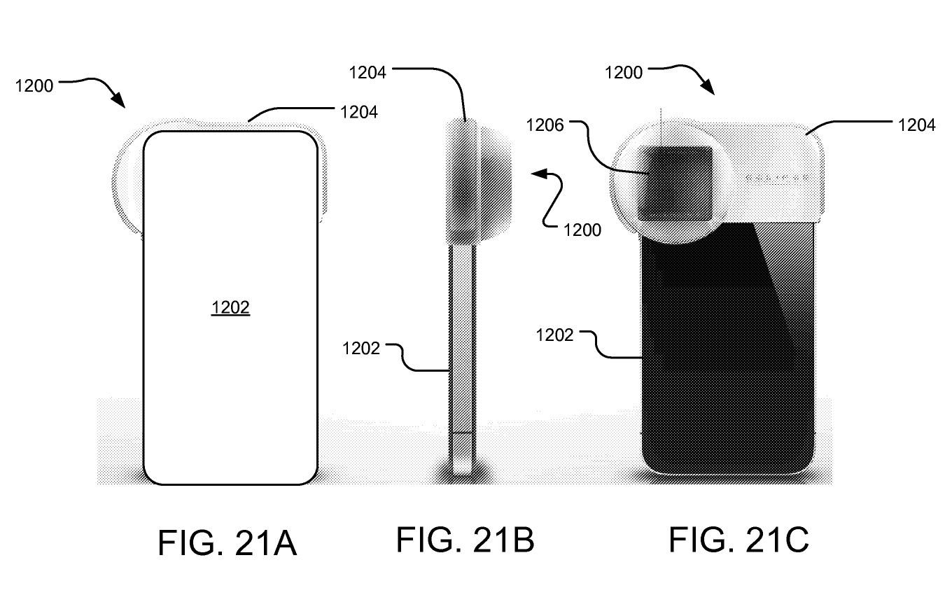

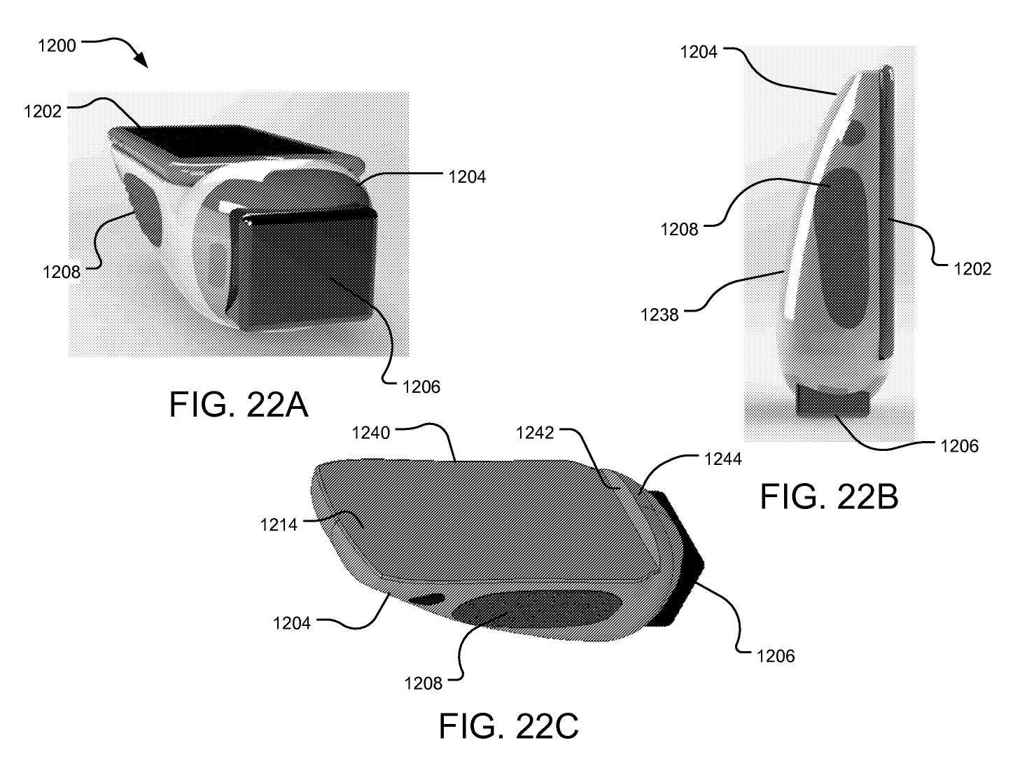

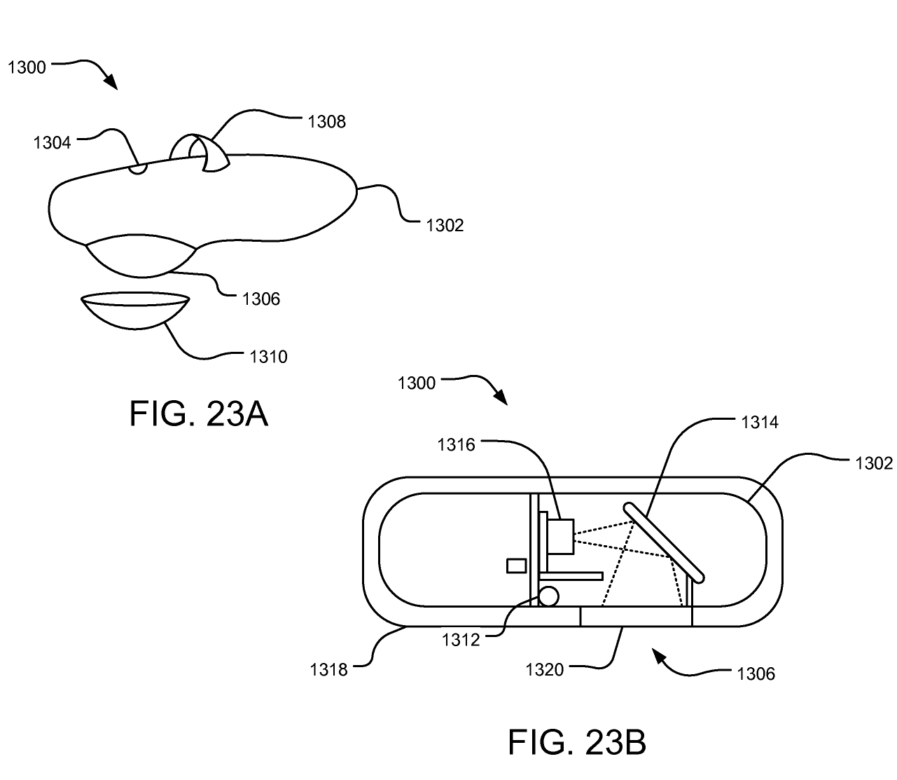

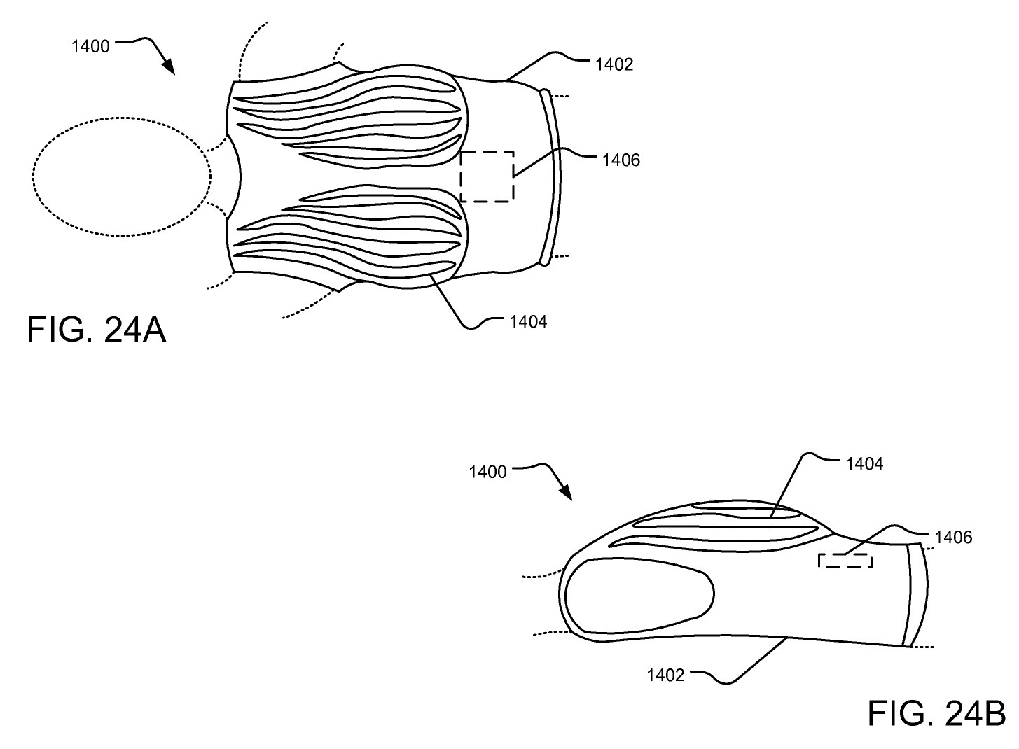

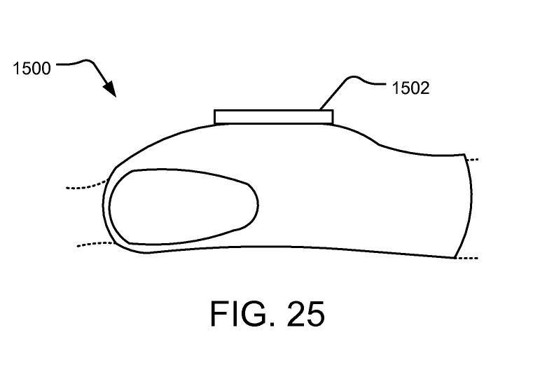

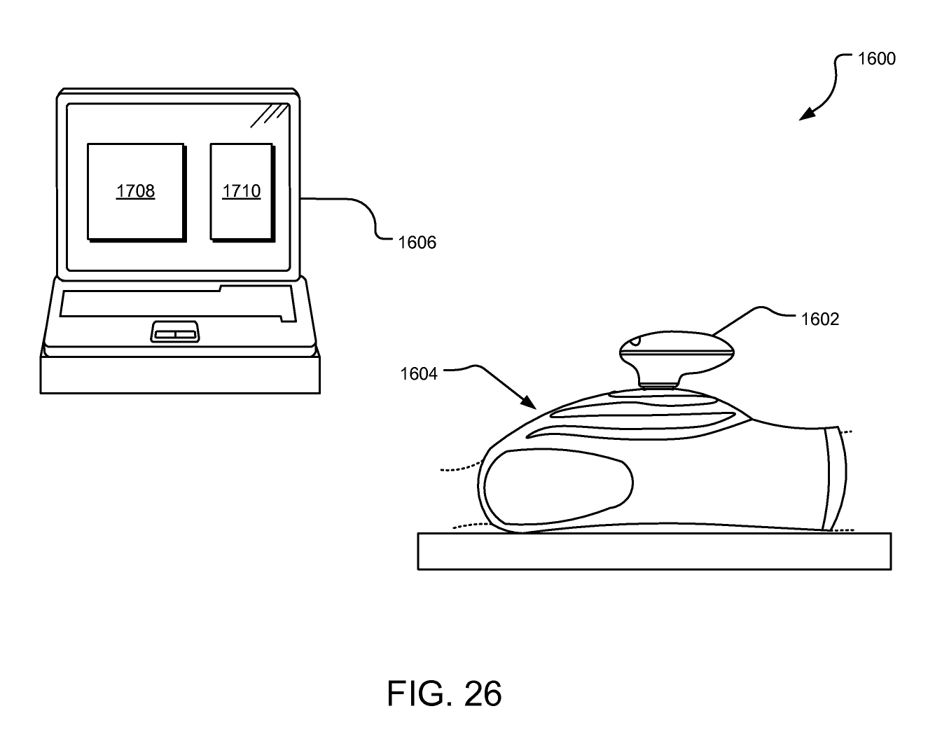

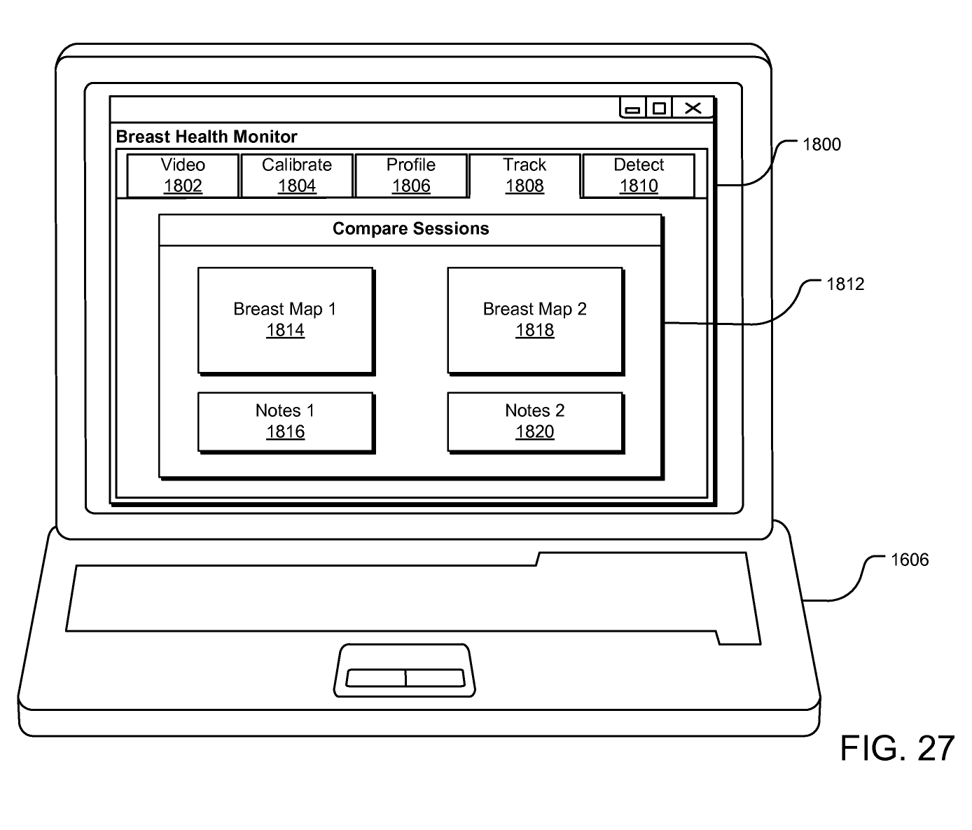

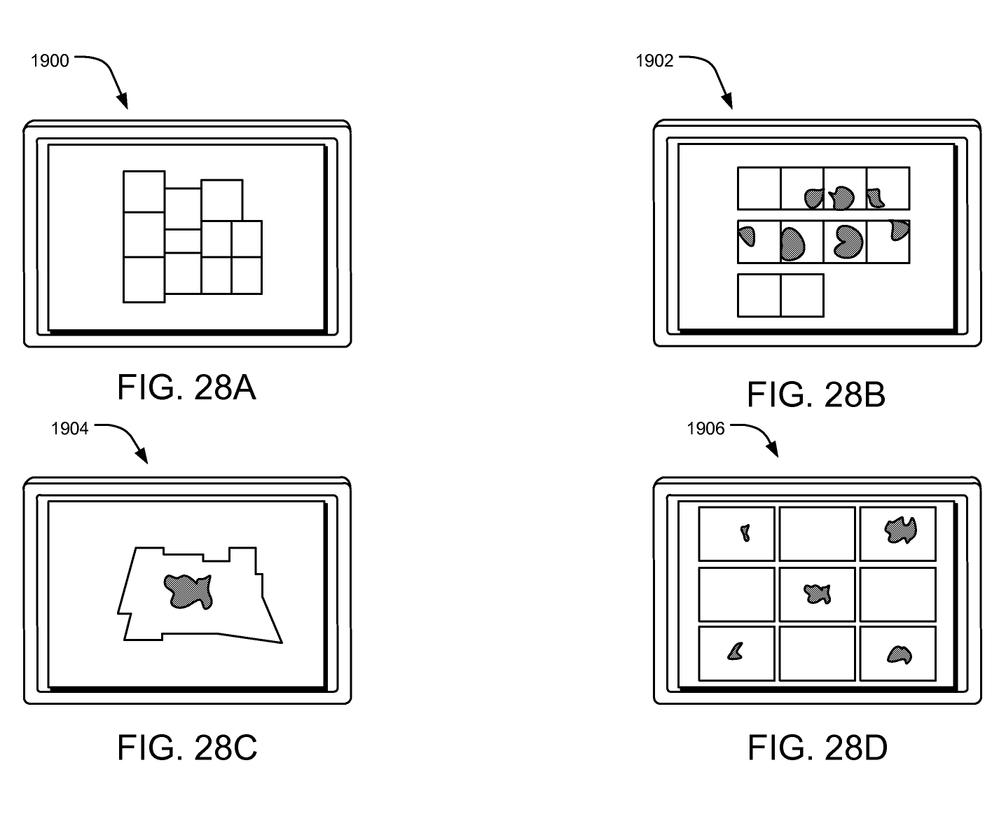

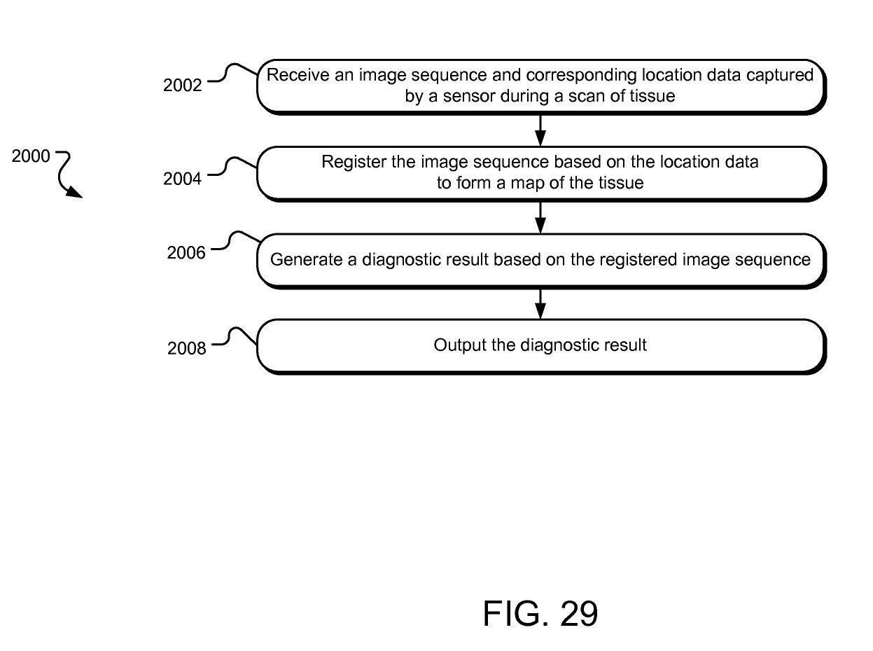

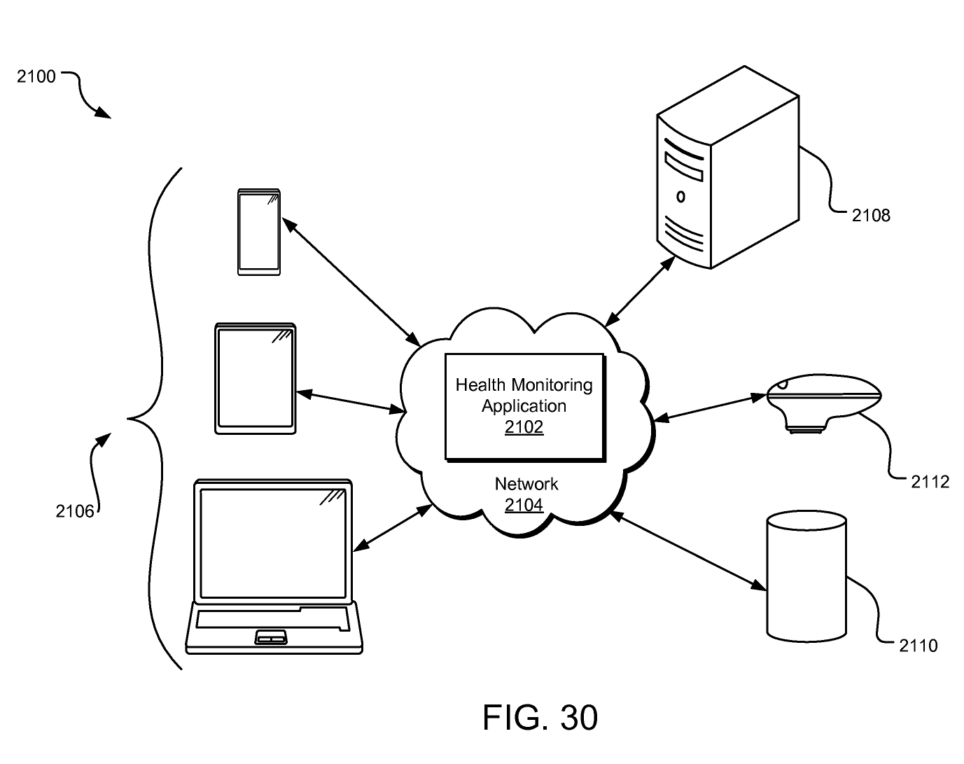

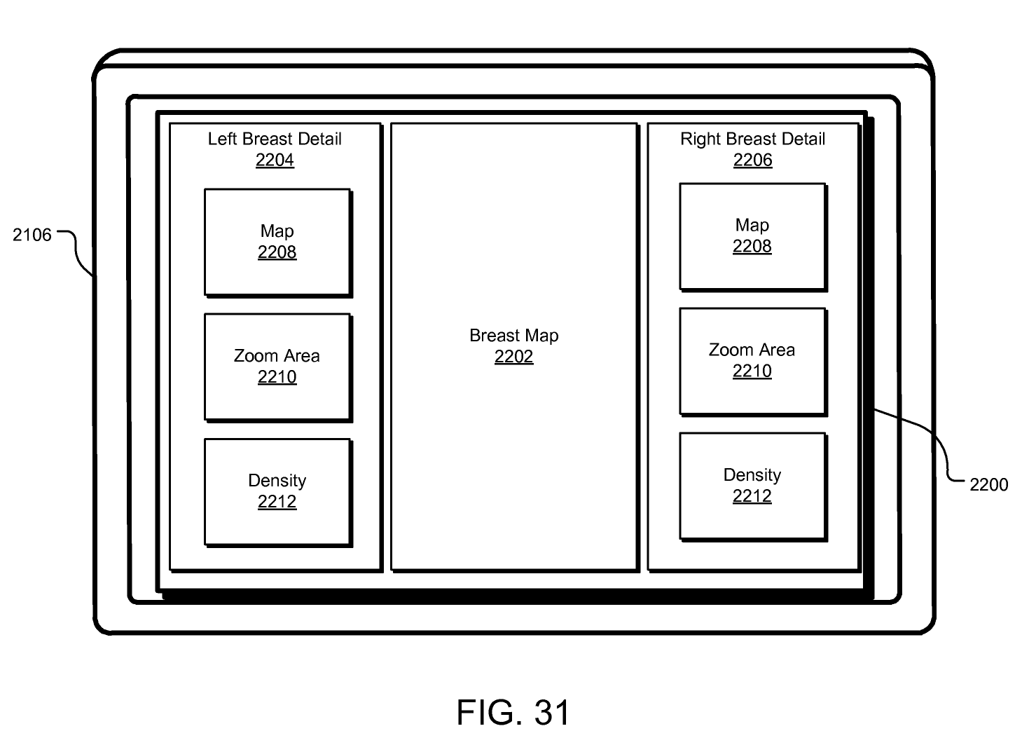

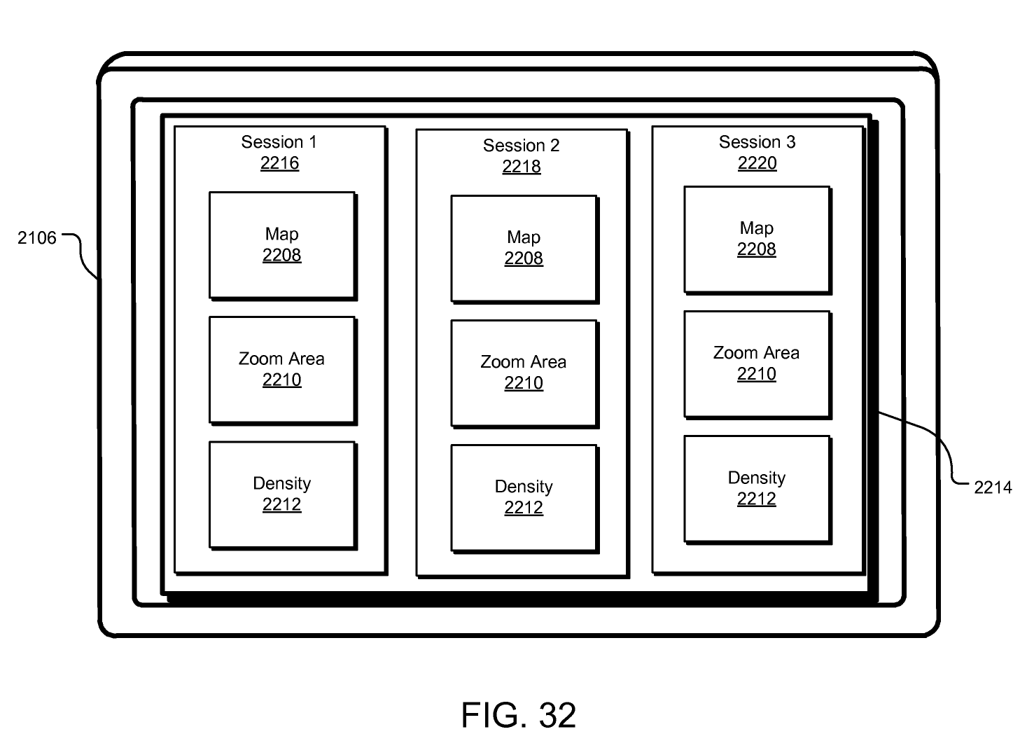

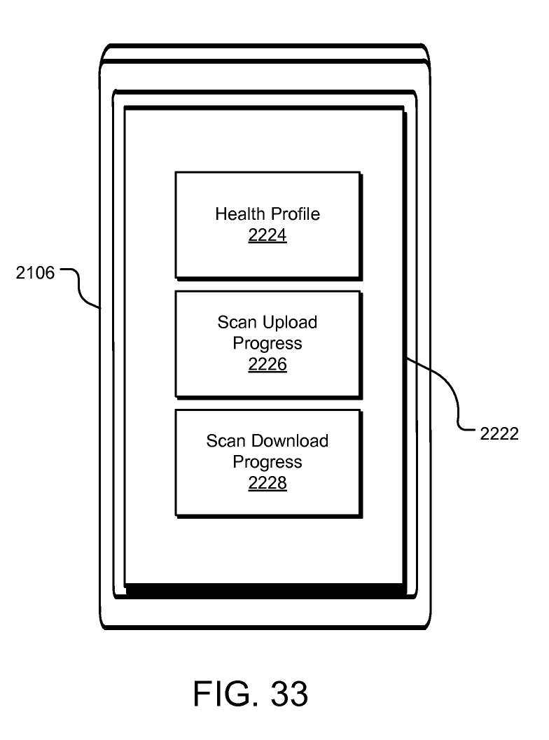

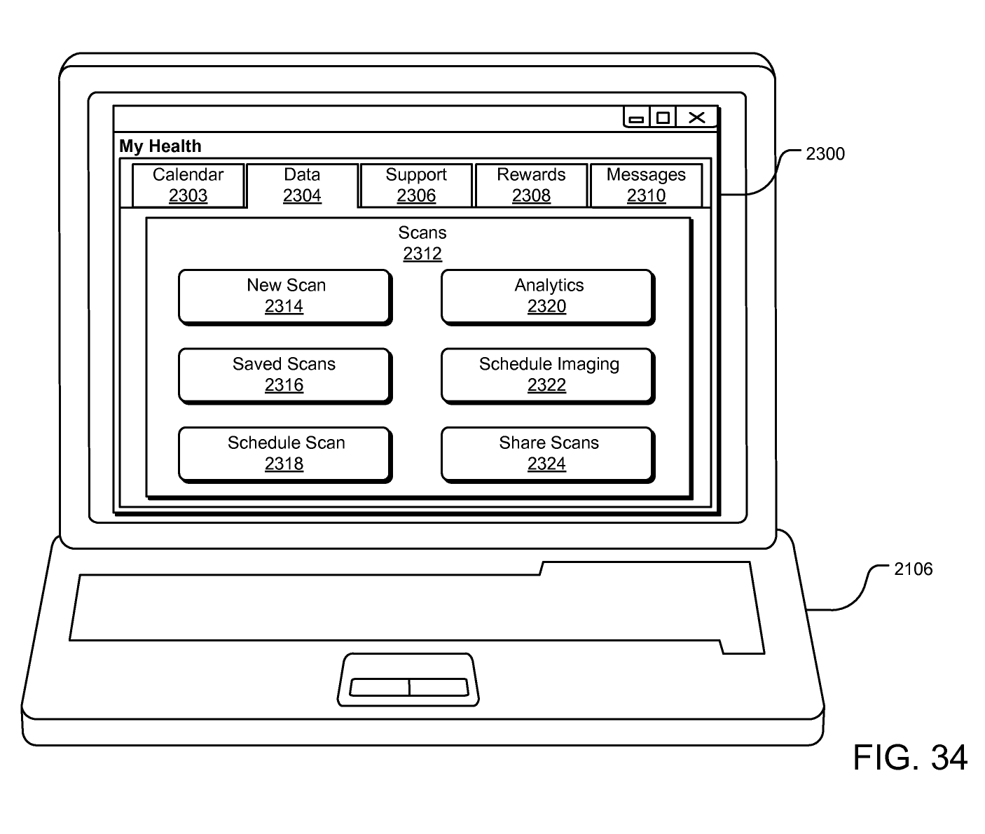

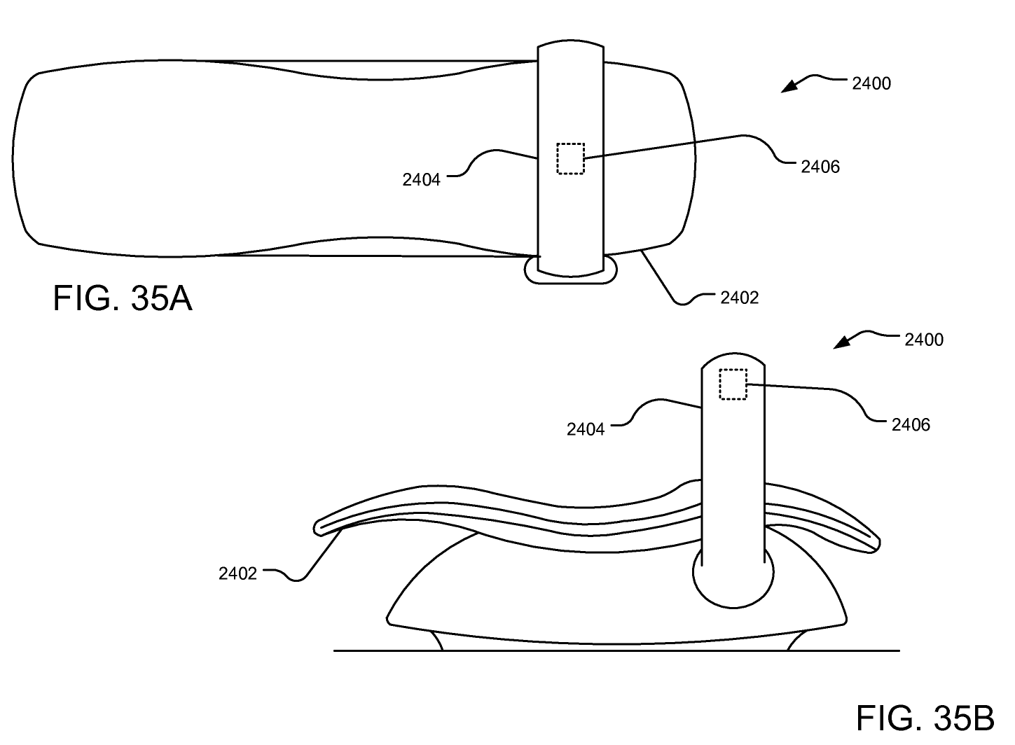

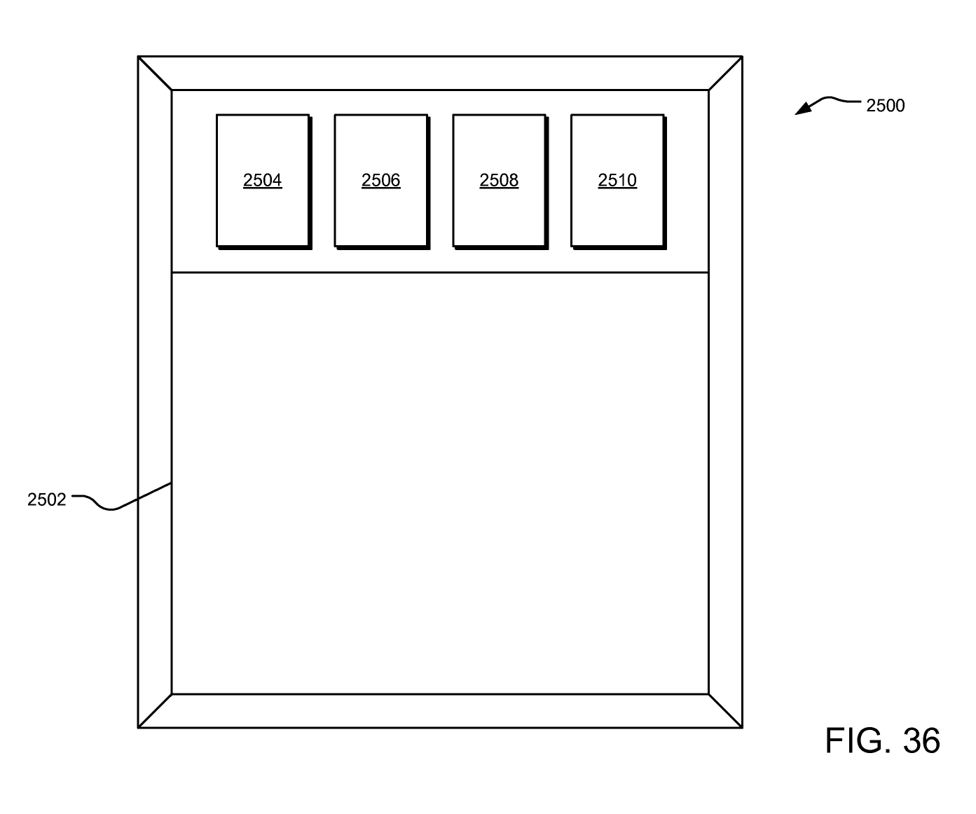

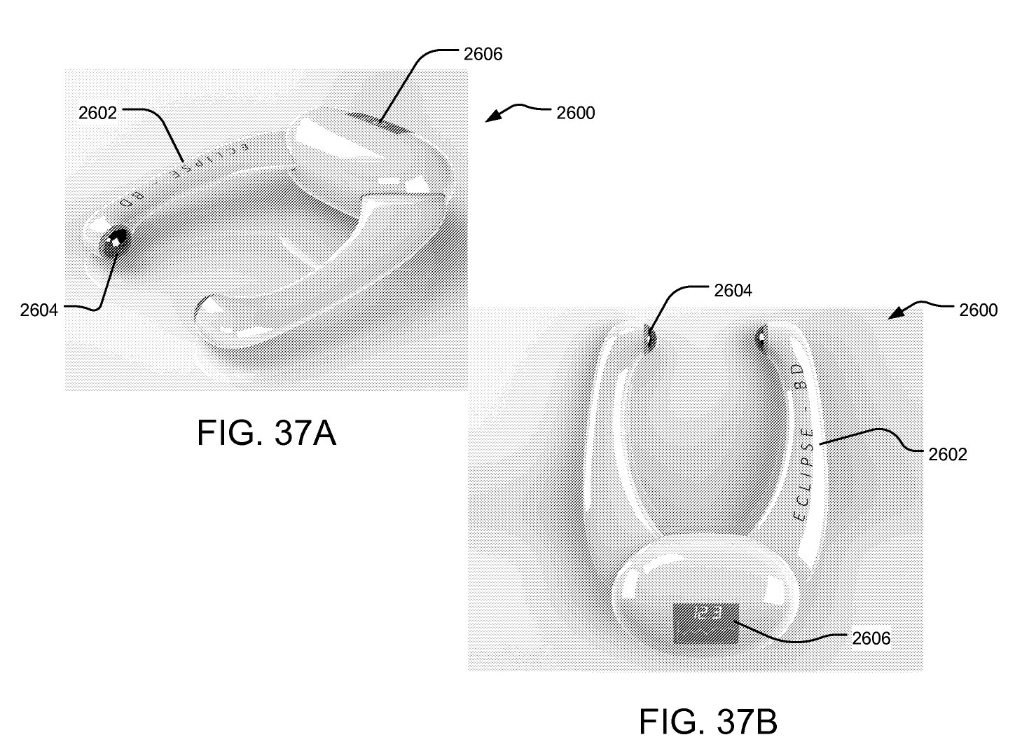

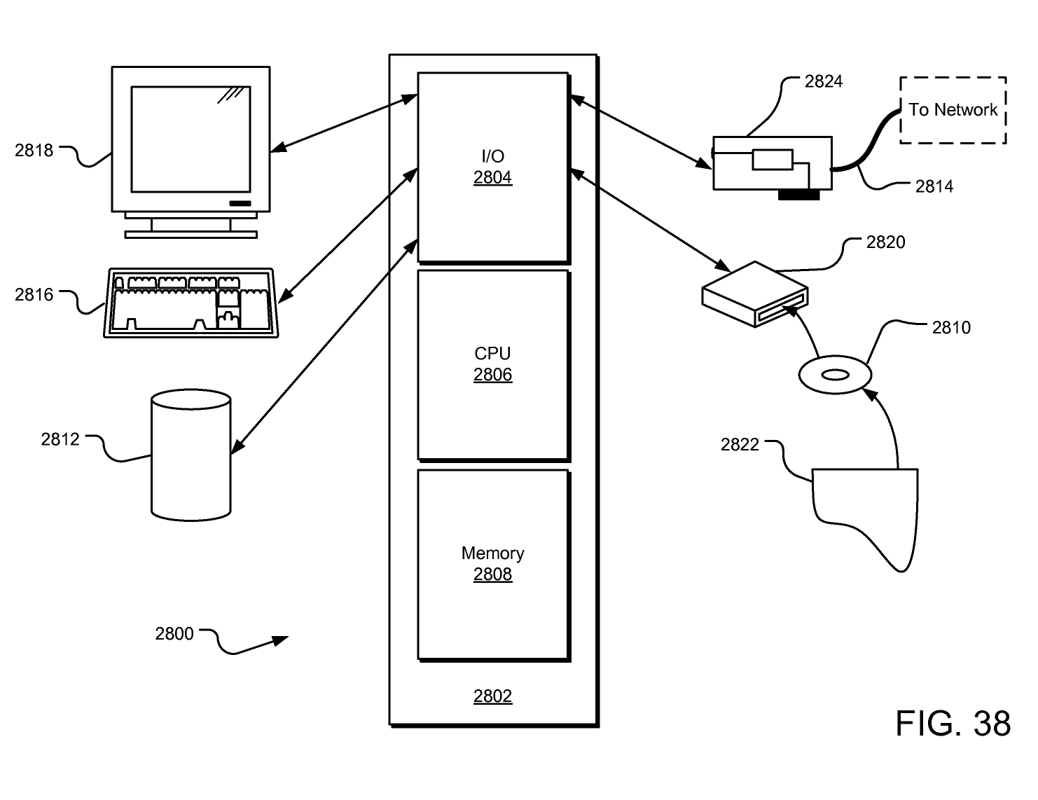

The sensor head 606 may include or more optical, tactile, and/or wave front sensors. For example, referring to FIGS. 10A-10B, the sensor head 606 may include: one or more light sources 616, such as LED's, diode lasers, organic LED's, suitably collimated incandescent light sources, and/or the like; an optical waveguide 618; an imaging array 620; and a PCB 622. In one implementation, the sensor head 606 includes a compliant surface configured to compress the target tissue to obtain data from scattered photons captured at the imaging array 620. In another implementation, the sensor head 606 transmits a wave front signal and receives a bounce-back signal captured at the imaging array 620. For example, the sensor head 606 utilize NIR optical sensors. As shown in FIG. 100, the sensor head 606 may include one or more mirrors 628 to redirect photons through collimating optics 630 into an imaging array 632. However, other sensor configurations are contemplated as described herein.

In one implementation, the sensor head 606 involves rocking and/or rolling actuation along the directions shown by the arrows in FIGS. 10A-10B, respectively. The sensor head 606 may be manually rolled over the target tissue manually by a user. In one implementation, the sensor head 606 automatically actuates, for example, using one or more motors 624. The device 600 has electronics 626 that may be used to control the operation of the device 600, including the actuation of the sensor head 606, the transmission and collection of signals and data, feedback to the user, and the like.

FIGS. 11A-11C illustrate another example health monitoring device 700 with rolling actuation. Similar to the various health monitoring devices described herein, the device 700 may include a body 702 and a sensor head 704. The sensor head 704 may include or more optical, tactile, or wave front sensors. In one implementation, the sensor head 704 includes: one or more light sources 706, such as LED's, diode lasers, organic LED's, suitably collimated incandescent light sources, and/or the like; an optical waveguide 708; an imaging array 710; and a PCB 712. However, other sensor configurations are contemplated as described herein.

FIGS. 12A-14B depicts various views of an example health monitoring device 800 with disposable, interchangeable, reversible, or otherwise removable sensor heads. In one implementation, the device 800 includes a body 802 having one or more contoured portions 804, a sensor head 806, one or more control buttons 810, an on/off button 830, a user interface 812, and grips 808 along the contoured portions 804 of the body 802. In one implementation, the sensor head 806 involves rocking and/or rolling actuation, with the rocking actuation along the direction of the arrow shown in FIG. 12C.

The sensor head 806 may include or more optical, tactile, or wave front sensors. As shown in FIG. 13A, in one implementation, the sensor head 806 may include: a sensor cover 816; one or more sensors 814, for example, having light sources and an optical waveguide, or other sensor components configured to transmit various wave front signals. The device 800 may further include: one or more mirrors 818 configured to direct photons into an imaging array 820; electronics 822; a power source 824, such as a battery; a wireless link or wired connector 826; a charging coil 828; and an on/off button 830. As shown in FIG. 13C, the sensor head 806 may have a variety of shapes and be configured for actuation in various manners. For example, the sensor head 806 may have a mount 832 engaged to a sensor 834 configured to move within the mount 832.

Turning to FIGS. 14A-14B, in one implementation, the body 802 includes a first cover 836 configured to engage a second cover 838 at an engaging portion 840 to enclose the body 802 to form an interior housing. In one implementation, the covers 836 and 838 may be removable to disassemble the device 100 to facilitate replacement, disposal, cleaning, and/or upgrade of the components of the device 800. The cover 836 may include a protruding portion 842 extending outwardly from a body of the cover 836 and defining an opening 844 through which one or more wave front signals may be transmitted and read back.

In one implementation, a docking station 846 is adapted to receive the device 800. The docking station 846 may include a body 848 having a receiving portion 850 may be sized and shaped to receive the protruding portion 842. The docking station 842 may charge the device 800 through power drawn from a power supply. The docking station 842 may be further configured to collect data from the device 800 and transmit the data via a wired or wireless connection 852 to a computing device and/or over a network.

The device 800 may include various removable, disposable, and/or interchangeable sensors and/or sensor heads 806 that may be utilized based on the operation of the device 800. In one implementation, the user interface 812 provides feedback to the user and includes one or more options for controlling the operation of the device 800. In one implementation, the user interface 804 includes a visual digital readout and/or other components for providing feedback, such as a speaker to provide audio feedback or light sources to provide other visual feedback. For example, the user interface 804 may provide sound indicators associated with saturation, movement, user instructions for operations, results, alerts or reminders, status (e.g., uploading scan data, completing a scan, etc.), location, orientation, maintenance, and the like.

FIG. 15 shows a side view of another example health monitoring device 900. In one implementation, the device 900 includes a body 902 having a handle 904, a sensor head 906, an on/off button 908, and a user interface 910.

The handle 904 of the body 902 may be sized and shaped to comfortably fit in a hand of a user. The handle 904 may have various surface features, angles, and/or contours to facilitate use and enhance comfort. For example, as shown in FIG. 15, the handle 904 may have surface contours for easy gripping by a hand of a user.

The user interface 910 provides feedback to the user and includes one or more options for controlling the operation of the device 900, including actuation of the sensor head 906. In one implementation, the sensor head 906 involves rolling actuation. The sensor head 906 may include or more optical, tactile, or wave front sensors. However, other sensors as described herein are contemplated.

Referring to FIGS. 16A-16C, an example round health monitoring device 1000 is shown. In one implementation, the device 1000 includes a body 1002, a docking station 1004, a sensor head 1006, an on/off button 1008, and a user interface 1010.

In one implementation, the body 1002 has a rounded shape sized to comfortably fit in a hand of a user. The docking station 1004 is adapted to receive the device 1000. The docking station 1004 may charge the device 1000 through power drawn from a power supply. The docking station 1004 may be further configured to collect data from the device 1000 and transmit the data via a wired or wireless connection to a computing device and/or over a network.

The user interface 1010 provides feedback to the user and includes one or more options for controlling the operation of the device 1000. In one implementation, the user interface 1010 includes a visual digital readout and/or other components for providing feedback, such as a speaker to provide audio feedback or light sources to provide other visual feedback. In one implementation, the user interface 1010 includes a translucent surface through which the feedback is provided.

In one implementation, the sensor head 1006 involves gliding or pressing actuation. The sensor head 1006 may include or more optical, tactile, or wave front sensors. However, other sensors as described herein are contemplated. For example, referring to FIG. 16C, the device 1000 may include: one or more light sources 1012, such as LED's, diode lasers, organic LED's, suitably collimated incandescent light sources, and/or the like; an imaging array 1014; and electronics 1016. In one implementation, the sensor head 1006 includes a compliant surface configured to compress the target tissue to obtain data from scattered photons captured at the imaging array 1014. In another implementation, the sensor head 1006 transmits a wave front signal and receives a bounce-back signal captured at the imaging array 1014. However, other sensor configurations are contemplated as described herein.

FIGS. 17A-17B illustrate an example finger loop health monitoring device 1100 with force activation. In one implementation, the device 1100 includes: a body 1102 with an opening 1104 configured to receive fingers of a user; a sensor head 1106; and an on/off button 1108.

In one implementation, the device 1100 is activated for a scan with an application of a minimum threshold of force to the sensor head 1106. The minimum threshold of force may be, for example, approximately 5 pounds of force. The sensor head 1106 may include or more optical, tactile, or wave front sensors. However, other sensors as described herein are contemplated.

FIGS. 18A-22C show various example digital health monitoring devices 1200 configured to operate using a portable user device 1202, such as a smartphone, tablet, and/or the like. Generally, the device 1200 includes: a body 1204 configured to removably engage and communicate with the portable user device 1202; a sensor head 1206; and one or more grips 1208. The sensor head 1206 may include or more optical, tactile, or wave front sensors. However, other sensors as described herein are contemplated. The device 1200 may be configured to communicate with the user device 1202 via a wired connection (e.g., USB connection) and/or a wireless connection (e.g., Bluetooth connection).

The body 1204 may have a variety of shapes and sizes configured to facilitate use and communication with the user device 1202. The body 1204 may further include various designs, textures, surfaces, portions, and/or other aesthetic features. It will be appreciated that the designs of the device 1200 shown in FIGS. 18A-22C are exemplary only and not intended to be limiting.

Turning first to FIGS. 18A-18C, in one implementation, the body 1204 includes a sleeve 1210 configured to receive and hold the user device 1202. The sleeve 1210 may be sized and shaped to receive and engage a variety of computing devices 1202. In one implementation, the sleeve 1210 is formed from one or more engaging surfaces 1214 with one or more lips 1216 extending therefrom to a rim 1218. In some implementations, the body 1204 includes one or more adjustable sections to customize the sleeve 1210 for engaging different computing devices 1202. Moreover, it will be appreciated that the sleeve 1210 may have a variety of shapes and sizes. Similarly, a projecting portion 1212 extending outwardly from the body 1204 in a direction opposite the sleeve 1210 to the sensor head 1206 may have a variety of shapes, sizes, and aesthetic features. In some implementations, the projecting portion 1212 supports the sensor head 1206.

As can be understood from FIGS. 19A-19B, in one implementation, the sleeve 1210 is defined by a proximal portion 1220 connected to a peripheral portion 1222, and the projecting portion 1212 includes a neck 1224 extending from a surface 1228 to support a ring 1226. The sensor head 1206 is supported by and positioned in the ring 1226. Referring to FIGS. 20A-20B, in one implementation, the sleeve 1210 is sized and shaped to receive an entirety of the user device 1202, as opposed to a portion as shown, for example, in FIGS. 18A-18C and FIGS. 21A-C. To securely engage the user device 1202 within the sleeve 1210, the rim 1218 extends around the sleeve 1210 between edges 1236. In one implementation, the body 1204 may include one or more contoured surfaces, for example, to form a pinched waist 1234 to facilitate use.

As can be understood from FIGS. 22A-22C, in one implementation, the body 1204 includes a contoured surface 1238 disposed opposite the engaging surface 1214 and tapering along a length 1240 of the body 1204 moving away from a base 1244. The base 12144 may include an edge surface 1242 defined therein and configured to receive an edge of the user device 1202. In one implementation, the engaging surface 1214 extends along the length 1240 of the body 1204 from the edge surface 1242. The engaging surface 1214 may extend at a variety of angles. For example, the engaging surface 1214 may have an incline of approximately five degrees.

FIGS. 23A-23B illustrate an example health monitoring device 1300 for use in spa, beauty, or wellness settings. In one implementation, the device 1300 includes: a body 1302, a user interface 1304, a sensor head 1306, a hand loop 1308, and a touch-down pad 1310.

In one implementation, the touch down pad 1310 protects the sensor head 1306 to permit the device 1300 to be used with creams, gels, soaps, lotions, oils, or the like, for example, in the shower or bath. The use of such skincare products facilitates sliding and movement of the sensor head 1306 against the skin during a scan and also encourages the use of the device 1300 during a regular wellness or beauty routine of a user. In one implementation, the device 1300 includes a membrane 1318 that may distribute skincare products and/or protect the device 1300 from moisture and other foreign particulates.

The sensor head 1306 may include or more optical, tactile, or wave front sensors. For example, referring to FIG. 23E, the device 1300 may include: one or more light sources 1312, such as LED's, diode lasers, organic LED's, suitably collimated incandescent light sources, and/or the like; an optical waveguide 1320; and one or more mirrors 1314 configured to direct a signal at an imaging array 1316. In one implementation, the sensor head 1306 includes a compliant surface configured to compress the target tissue to obtain data from scattered photons captured at the imaging array 1316. In another implementation, the sensor head 1306 transmits a wave front signal and receives a bounce-back signal captured at the imaging array 1316. However, other sensor configurations are contemplated as described herein.

As described herein, in some implementations, the health monitoring device includes or operates in conjunction with one or more target enhancements to facilitate signal transmission and receipt. Such target enhancements may include, without limitation, touch-down pads with various geometries, textures, and/or materials; mechanical enhancements, such as waveguide and/or sonic enhancements; conductive materials, such as gels and/or pressure plates; compression enhancements, including movement dynamics orientation; placement enhancements, for example, involving gravity, magnetics, and/or electro-mechanical aspects; automation, including robotics, stabilization, and/or vibration; and/or thermal enhancements, including photonic and/or electronic. FIGS. 24A-25 show example implementations of such target enhancements. It will be appreciated, however, that these examples are intended to be illustrative rather than limiting.

Turning first to FIGS. 24A and 24B, it will be appreciated that the quality of collected sensor data, such as image data, may be enhanced by placing a coupling material 1400 between a health monitoring device and the target tissue. The coupling material 1400 may be, for example, a garment 1402 or a disposable or impressionable object. The coupling material 1400 may provide stabilization to the target tissue during the exam, for example, with a stiff or firm fabric or a reinforced fabric structure.

As can be understood from FIGS. 24A and 24B, in one implementation, the coupling material 1400 includes a guide pattern 1404, which provides the user with a diagram of an appropriate scan routine to follow for a particular exam. The example guide pattern 1404 shown in FIGS. 24A and 24B may be used during a breast exam. The guide pattern may be visible or may be hidden until prompted by the device. For example, at least a portion of the guide pattern 1404 may be illuminated with specific radiation emitted from the device during a scan or may become visible when pressure is exerted against the guide pattern 1404 during the scan.

In one implementation, the garment 1402 includes one or more sensors 1406 for performing manual or fully automated scans of target tissue. The sensors 1406 may include any of the sensors described herein.

The garment 1402 may press the sensors 1406 against the target tissue (e.g., the breasts). As the sensors 1406 move relative to the target tissue, the sensors 1406 collect data for analysis. A pillow or cushioning object may similarly perform exams using one or more sensors like the sensors 1406.

Turning to FIG. 25, an example conductive material 1500 to facilitate signal transmission and receipt by a health monitoring device is shown. In one implementation, the material 1500 is a plate 1502 that may be used as a sensor head to image or manipulate larger tissue areas. The plate 1502 may be used with a manual device or with an automated device employing robotics. The plate 1502 may be used with various imaging techniques, as described herein, including without limitation, tactile, thermal, and optical.

FIG. 26 depicts an example system 1600 for health monitoring, including a health monitoring device 1602 used with a target enhancement 1604 (e.g., a garment) in communication with a user device 1606, which may be any form of computing device as described herein.

In one implementation, the device 1602 includes a body housing one or more sensors mounted with a strain gauge on a mount plane. The sensors may include one or more light sources, an optic waveguide and wave front channel, an electromagnetic and/or mechanical wave front generator, an optic filter, a photonic capture or transfer plane, and an image array (e.g., a high resolution CCD camera). The sensors may additional include a translucent touch down pad (e.g., made from silicone) and electronics configured to output the scan data to the user device 1606.

After initiating a scan, in one implementation, the device 1602 transmits a wave front signal into the target tissue using, for example, a combination of electro-optical and electromechanical signals using pulsed modulation. The device 1602 receives and interprets the bounce-back signal. In one implementation, the harder the tissue, the higher the wave-frequency. The data is then output to the user device 1606 for processing. In one implementation, a scanning application running on the user device 1606 filters and discriminates the image for interpretation review.

A scan may involve a random and continuous movement of the device 1602 over the target tissue. For example, the motion may resemble a painting or scanning motion. The scanning application running on the user device 1606 identifies and fills in blanks in the scan data while stitching the images together based on the location and orientation of the device 1602. The device 1602 and/or the user device 1606 may provide alerts or cues, for example, through sound, vibrations or visuals, to indicate a status of the scan and when the target area has been covered. A starting point of the scan may be recognized by tracking imager or sensor or may be base-lined by a visual point (e.g., a nipple, skin recognition patter) or by durometer or other physical points in the anatomy like a collar bone. The scanning application may utilize MEM and visual coordinates to automatically stitch or otherwise assembly individual snapshots of target tissue into a full map of the target tissue. In one implementation, the scanning application displays one or more maps 1708 and/or calibration options 1710 via a user interface.

For an example of such a user interface, reference is made to FIG. 27, which shows an example breast health monitor user interface 1800. It will be appreciated that the user interface 1800 is exemplary only and not intended to be limiting. In one implementation, the user interface 1800 includes various tabs 1802-1810 for navigating to different resources for breast health monitoring. For example, a video tab 1802 may be used to display a video of a scan, a calibrate tab 1804 may be used to calibrate one or more devices 1602, a profile tab 1806 may be used to obtain information on one or more users, a tracking tab 1808 may be used to view and compare scans, and a detect tab 1810 may be used to initiate and operate the device 1602 during a scan. It will be appreciated, however, that more or fewer tabs may be used for navigation.

In one implementation, selection of the tracking tab 1808 will present a compare sessions window 1812 displaying a first breast map 1814 and associated notes 1816 corresponding to a first session for comparison to data from one or more other sessions, such a second breast map 1818 and associated notes 1820 corresponding to a second session. The breast maps 1814 and 1818 may be displayed with a grid to locate any potentially problematic areas and with color coding indicating a tissue hardness to facilitate the tracking of any changes and the identification of any concerning areas.

To understand the capture, alignment, and processing of scans for early diagnosis of diseases and conditions, reference is made to FIGS. 28A-29. Turning first to FIGS. 28A-28D, in one implementation, the captured images are rendered, aligned, stitched, and presented as a map, as shown in user interfaces 1900-1906, respectively. Referring to FIG. 29, example operations 2000 for noninvasive detection and early diagnosis of diseases and conditions. In one implementation, a receiving operation 2002 receives an image or an image sequence and corresponding location data captured by a sensor during a scan of tissue by a monitoring device. Each of the images received during the receiving operation 2002 is created by pressing the monitoring device against a surface of the tissue and corresponds to the pressure of the underlying tissue. As such, if a lump, lesion, or other hard abnormality is present in the tissue, the corresponding image received during the receiving operation 2002 includes an element that is represented as harder than surrounding tissue. The image sequence and corresponding location data receiving during the receiving operation 2002 may be pre-filtered prior to processing.

A registering operation 2004 registers or stiches the image sequence together based on the location data to form a map of the tissue. The individual images or map of the tissue may be transmitted for storage and/or subsequent review by a user, such as a patient or healthcare provider. In one implementation, the registering operation 2004 uses processing algorithms and/or image data mining algorithms, such as Monte Carlo or other simulations.

A generating operation 2006 generates a diagnostic result based on the registered image sequence. The diagnostic result may include a determination of the presence or absence of any abnormalities. In one implementation, the generating operation 2006 generates the diagnostic result using direct detection. In another implementation, the generating operation 2006 generates the diagnostic result using image alignment algorithms that compare the registered image sequence to images from prior exams to identify any deltas representing changes of the target tissue. In still another implementation, the generating operation 2006 generates the diagnostic result using image reconstruction and filtering.

An outputting operation 2008 outputs the diagnostic result. In one implementation, the outputting operation 2008 transmits the diagnostic result to a user, such as the patient, a healthcare provider, or the like for review. In another implementation, the outputting operation uploads the diagnostic result for storage in an online repository or other database.

FIG. 30 is an example health monitoring system 2100 for routine health monitoring and noninvasive detection and early diagnosis of diseases and conditions is shown. In the implementation, a user, such as a patient, healthcare provider, or other interested party, accesses and interacts with a health monitoring application 2102 via a network 2104 (e.g., the Internet).

The network 2104 is used by one or more computing or data storage devices (e.g., one or more databases 2110) for implementing the health monitoring system 2100. The user may access and interact with the health monitoring application 2102 using a user device 2106 communicatively connected to the network 2104. The user device 2106 is generally any form of computing device capable of interacting with the network 2104, such as a personal computer, workstation, terminal, portable computer, mobile device, smartphone, tablet, multimedia console, etc.

A server 2108 hosts the system 2100. The server 2106 may also host a website or an application, such as the health monitoring application 2102 that users visit to access the system 2100. The server 2106 may be one single server, a plurality of servers with each such server being a physical server or a virtual machine, or a collection of both physical servers and virtual machines. In another implementation, a cloud hosts one or more components of the system 2100. One or more health monitoring devices 2112, the user devices 2106, the server 2106, and other resources, such as the database 2110, connected to the network 2104 may access one or more other servers for access to one or more websites, applications, web services interfaces, etc. that are used for routine health monitoring and noninvasive detection and early diagnosis of diseases and conditions. The server 2106 may also host a search engine that the system 2100 uses for accessing and modifying information used for health monitoring and noninvasive detection and early diagnosis of diseases and conditions.

In another implementation, the user device 2106 locally runs the health monitoring application 2104, and the monitoring devices 2112 connect to the user device 2106 using a wired (e.g., USB connection) or wireless (e.g., Bluetooth) connection.

Using the health monitoring application 2102, a user may upload health information, including history and information corresponding to any prior exams. The health monitoring application 2102 may generate reminders to prompt a patient to obtain an exam at regular or random intervals, dictate real-time instructions for the use of the monitoring device 2112, and/or other tasks. The health monitoring application 2102 may record a user's verbal or written notations during an exam using sensors in the monitoring device 2112 and/or the user device 2106.

In one implementation, the health monitoring application 2102 includes various instructions for processing health information based on the type of data provided by the monitoring device 2112. Stated differently, the health monitoring application 2102 may process health information based on the type of sensor utilized by the monitoring device 2112 during an exam.

For example, the monitoring device 2112 may be used to collect a sequence of images at a reasonably fast rate (e.g., approximately ten frames per second) while simultaneously tracking the relative location and orientation of each subsequent image. The monitoring device 2112 tags the images with such metadata, enabling the health monitoring application 2102 to determine the overlap between any two images in the acquired image sequence. In one implementation, the health monitoring application 2102 pre-filters the individual images to enhance properties of the images, such as contrast and overall intensity.

The health monitoring application 2102 stiches the images together to form a map or composite image of the examined tissue, such as a breast. To create an accurate composite image, the health monitoring application 2102 may perform functions, including, without limitation, intensity averaging, stretching or other diffeomorphisms (particularly to accommodate variations in perspective), phase correlation, application of a nonlinear color space, frequency-domain processing, feature identification, conversions to different coordinate systems (e.g., log-polar coordinates), and other manipulations. The health monitoring application 2102 may process the composite image using algorithms, such as Monte Carlo or other simulation techniques, to translate the composite image into one or more different formats, such as an accurate visual representation of the scanned tissue. A visually realistic representation may incorporate not only restructuring of the intensity pattern, but also the elimination of visually detracting artifacts, such as Mach bands or haloing.

Once the health monitoring application 2102 processes and analyzes health information corresponding to an exam of target tissue, the user may utilize the health monitoring application 2102 to perform various functions. For example, the health monitoring application 2104 may perform image feature identification to flag potentially problematic areas in the examined tissue that may need follow-up testing. The health monitoring application 2104 may perform such functions automatically or upon a command from a user. Further, the health monitoring application 2104 may compare a new image to images collected during other scans, taken at various times and/or with various sensors or other equipment (e.g., x-ray machine) to determine whether any significant changes occurred. In one implementation, the health monitoring application 2104 performs image manipulation, registration, and/or differencing to align the images for comparison. Based on the comparison or direct analysis, the health monitoring application 2104 generates a diagnostic result.

In one implementation, a user, such as the patient, downloads the diagnostic result to the user device 2106, which the patient may bring to discuss with a healthcare provider. In another implementation, the health monitoring application 2104 automatically or upon a command from the user submits a prompt to seek for review by a medical professional that may lead to diagnosis or the diagnostic result to the patient's healthcare provider over the network 2102. The diagnostic result may include an identification of any watch spots, problem spots, recommendations for follow-up exams, such as a mammogram, and/or other analysis. The scans, diagnostic results, exam results, and/or any other health information may be stored in the database 2110, which may be accessed by a user with the health monitoring application 2104.

FIGS. 31-34 illustrate additional example user interfaces generated by the health monitoring application 2104 and displayed in a window (e.g., a browser window) of the user device 2106.

Referring to FIG. 32, a map interface 2200 showing a breast map 2202 depicting, for example, right breast map and a left breast map generated based on scans taken on a particular date. The interface 2200 provides visual cues (e.g., color coding) to indicate a minimum and maximum hardness in the scanned target tissue, an average hardness, and a difference in hardness from previous scans. A more detailed view may be shown with left breast detail 2204 and right breast detail 2206, each displaying a detailed breast map 2208. A user may use a touch screen or other user input to display zoom areas 1210 of the breast maps displayed and obtain additional data about the current scan or comparison data from previous scans. Density detail 2212 may indicate a minimum and maximum hardness in the scanned target tissue, an average hardness, and a difference in hardness from previous scans for the zoom area 2210. Referring to FIG. 32, a comparison interface 2214 provides similar data with data for a plurality of sessions (e.g., sessions 2216-2220) for a breast shown side by side for a comparison.

FIG. 33 shows a health monitoring resources user interface 2222 provided, for example, on a mobile user device 2106, and displaying a health profile 2224 of the user, including previous scans, and a status 2226 of uploading the scans and a status 2228 of downloading the scans from various dates to the health monitoring application 2102.

FIG. 34 illustrates a health user interface 2300 displaying various health monitoring resources. The interface 2300 may include one or more tabs 2303-2310 providing access to different health resources. It will be appreciated that more or fewer tabs may be included, and the example shown in FIG. 34 is exemplary only and not intended to be limiting.

In one implementation, a calendar tab 2303 provides a schedule of health activities for the user, including, without limitation, imaging appointments, regular scans, medication taking, exercise or nutrition activities, appointments with medical professionals, reminders, and the like. A support tab 2306 provides access to resources, such as support groups, chat rooms, medical journals or articles, community information, social media, and the like. A rewards tab 2308 tracks and displays actions performed by the user that may trigger rewards to provide an incentive for completing healthy activities, such as scans. A messages tab 2310 collects and displays messages sent to and from the user, for example, from medical professionals, automatically or manually generated (e.g., providing data, receipts, prescriptions, instructions, etc.), related to social media, from friends or support groups, and the like.

A scan tab 2312 provides access to scans and resources involving scans. In one implementation, selection of the scan tab 2312 displays a scans window 2312 with options for initiating or uploading a new scan 2314, accessing saved scans 2316, scheduling a scan 2318, accessing analytics 2320 (e.g., comparisons, diagnoses, recommendations, etc.), scheduling an imaging appointment 2322 (e.g., a mammogram) with a medical professional, and sharing scans 2324 (e.g., sending the scans to a medical professional.

FIGS. 35A-37B illustrate additional devices similar to and including many of the same components as the devices described with respect to the preceding Figures and may be used with the health monitoring system 2100. It will be appreciated that other health monitoring devices may also be used with the system 2100 to monitor other conditions, for example, cancer, tissue density, body mass, temperature, blood oxygen, muscle mass, and/or the like.

Turning to FIGS. 35A and 35B, an example clinical health monitoring device 2400 is shown. In one implementation, the health monitoring device 2400 utilizes automated components and/or robotics. The relatively larger size of the device 2400 may produce higher resolution data, thereby increasing the quality of the exam results. In one implementation, the clinical device 2400 includes a table 2402 to receive a patient for an exam and an arm 2404 extending across the table 2402, such that a plane of the arm 2404 is generally parallel with a plane of the table 2402. The arm 2404 includes one or more sensors 2406. The sensors 2406 may include any of the sensors described herein. The health monitoring device 2400 may perform a non-touch automated image scan or a touch-down coupling contact or tactile scan.

In one implementation, the patient lies on the table 2402 with the target tissue positioned under the arm 2404. In another implementation, the patient lies on the table 2402 in any orientation, and the arm 2404 may be moved over the target tissue. During a scan, the target tissue is pressed against the arm 2404, for example, by raising the table 2402 to the arm 2404 or by lowering the arm 2404 against the target tissue. The scan is performed by moving and/or gyrating the device 2400, for example, using an actuator. The scan may be automated and/or controlled by a user, such as a technician or doctor. In one implementation, the arm 2404 includes one or more rollers to maintain a controlled pressure against the tissue during the exam, without causing discomfort to the patient.

Referring to FIG. 36, an example health monitoring device 2500 having a reflecting or digital mirror interface is shown. In one implementation, the mirror interface 2502 is a stationary screen-like display 2502 having one or more sensors 2504. The device 2500 may be used alone or in conjunction with other health monitoring devices, such as the handheld device 100. For example, the device 2500 may display a guide pattern layered over a real-time image of the target tissue of the patient for the patient to follow during an exam with the handheld device 2500.

The sensors 2504 may include any of the sensors described herein. For example, the sensors 2504 may include one or more passive sensors or thermal imaging sensors to monitor the patient's health, including, without limitation, body temperature, blood oxygenation, skin properties or lesions, internal tumors or lesions, heart rate, or other bodily functions and/or conditions. The device 2500 records such information using the sensors 2504 and may display the information to the patient in real time or other times on the display 2502.

In one implementation, the display 2502 includes a screen on the rear surface of a conventional reflecting mirror, such that the display 2502 functions as a conventional mirror having a reflective surface when the screen is off. In another implementation, the device 2500 is included as part of a larger apparatus containing mirrors, such as a medicine cabinet. In still another implementation, the device 2500 is a separate module that may be attached to a surface of a mirror. The device 2500 may be placed on a surface (e.g., counter) or mounted (e.g., similar to an articulating makeup mirror). In yet another implementation, the display 2502 is a digital mirror having a liquid crystal display (LCD) screen, or the like, and a camera for capturing an image for display on the screen. The device 2500 may include additional components for collecting data or providing benefits to the patient. For example, the device 2500 may include or be connected to a weight scale and/or contain illuminating sidebars to aid in application of beauty, health monitoring, or wellness products.

The device 2500 may be configured to perform exams in a variety of manners. In one implementation, the device 2500 may include a motion sensor for detecting the presence of a patient and automatically initiate an exam. In another implementation, the patient or other user may program the device 2500 to perform exams at specified regular intervals or upon the receipt of a command by the user. In still another implementation, the device 2500 may include communications 2506, including messages, alerts, reminders, and instructions, displayed on the display 2502 to prompt the patient to conduct an exam.

The device 2500 may include one or more modules 2508 for accessing a repository of the patient's health information, including without limitation: tactile, ultrasound, electro-optic, and other scans; visual or other representations of diagnostic results; tissue maps; written and verbal notes, recorded by the patient, healthcare provider, or other party; or the like. The modules 2508 may be used to display the health information to the patient on the display 2502. Further, the device 2500 may include one or more modules 2510 for performing additional functions. For example, the modules 2510 may be used to: send collected sensor data, pictures, video, or other health information to a healthcare provider over a network; communicate live with a healthcare provider over the network; delay the display of images of the patient to enable the viewing of body regions that the patient cannot see with a conventional mirror; or the like.

FIGS. 37A and 37B show an example tissue density monitoring device 2600. In one implementation, the device 2600 includes a body 2602, one or more sensors 2604, and a user interface 2606. The sensors 2604 may be configured to determine, without limitation, tissue (e.g., breast) density, body mass, temperature, blood oxygen, muscle mass, and/or the like using a tactile, optical, or wave front signal as described herein.

Referring to FIG. 38, a detailed description of an example computing system 2800 having one or more computing units that may implement various systems and methods discussed herein is provided. The computing system 2800 may be applicable to the user devices, the servers, the health monitoring devices, or other computing devices. It will be appreciated that specific implementations of these devices may be of differing possible specific computing architectures not all of which are specifically discussed herein but will be understood by those of ordinary skill in the art.

The computer system 2800 may be a general computing system is capable of executing a computer program product to execute a computer process. Data and program files may be input to the computer system 2800, which reads the files and executes the programs therein. Some of the elements of a general purpose computer system 2800 are shown in FIG. 41 wherein a processor 2802 is shown having an input/output (I/O) section 2804, a Central Processing Unit (CPU) 2806, and a memory section 2808. There may be one or more processors 2802, such that the processor 2802 of the computer system 2800 comprises a single central-processing unit 2806, or a plurality of processing units, commonly referred to as a parallel processing environment. The computer system 2800 may be a conventional computer, a distributed computer, or any other type of computer, such as one or more external computers made available via a cloud computing architecture. The presently described technology is optionally implemented in software devices loaded in memory 2808, stored on a configured DVD/CD-ROM 2810 or storage unit 2812, and/or communicated via a wired or wireless network link 2814, thereby transforming the computer system 2800 in FIG. 41 to a special purpose machine for implementing the described operations.

The I/O section 2804 is connected to one or more user-interface devices (e.g., a keyboard 2816 and a display unit 2818), a disc storage unit 2812, and a disc drive unit 2820. In the case of a tablet device, the input may be through a touch screen, voice commands, and/or Bluetooth connected keyboard, among other input mechanisms. Generally, the disc drive unit 2820 is a DVD/CD-ROM drive unit capable of reading the DVD/CD-ROM medium 2810, which typically contains programs and data 2822. Computer program products containing mechanisms to effectuate the systems and methods in accordance with the presently described technology may reside in the memory section 2804, on a disc storage unit 2812, on the DVD/CD-ROM medium 2810 of the computer system 2800, or on external storage devices made available via a cloud computing architecture with such computer program products, including one or more database management products, web server products, application server products, and/or other additional software components. Alternatively, a disc drive unit 2820 may be replaced or supplemented by an optical drive unit, a flash drive unit, magnetic drive unit, or other storage medium drive unit. Similarly, the disc drive unit 2820 may be replaced or supplemented with random access memory (RAM), magnetic memory, optical memory, and/or various other possible forms of semiconductor based memories commonly found in smart phones and tablets.

The network adapter 2824 is capable of connecting the computer system 500 to a network via the network link 2814, through which the computer system can receive instructions and data. Examples of such systems include personal computers, Intel or PowerPC-based computing systems, AMD-based computing systems and other systems running a Windows-based, a UNIX-based, or other operating system. It should be understood that computing systems may also embody devices such as terminals, workstations, mobile phones, tablets, laptops, personal computers, multimedia consoles, gaming consoles, set top boxes, and the like.

When used in a LAN-networking environment, the computer system 2800 is connected (by wired connection or wirelessly) to a local network through the network interface or adapter 2824, which is one type of communications device. When used in a WAN-networking environment, the computer system 2800 typically includes a modem, a network adapter, or any other type of communications device for establishing communications over the wide area network. In a networked environment, program modules depicted relative to the computer system 2800 or portions thereof, may be stored in a remote memory storage device. It is appreciated that the network connections shown are examples of communications devices for and other means of establishing a communications link between the computers may be used.

In an example implementation, health information, data captured by the one or more sensors, information collected by the monitoring devices, the health monitoring application 2104, a plurality of internal and external databases (e.g., the database 2110), source databases, and/or data cache on cloud servers are stored as the memory 2808 or other storage systems, such as the disk storage unit 2812 or the DVD/CD-ROM medium 2810, and/or other external storage devices made available and accessible via a cloud computing architecture. Health monitoring software and other modules and services may be embodied by instructions stored on such storage systems and executed by the processor 2802.

Some or all of the operations described herein may be performed by the processor 1002. Further, local computing systems, remote data sources and/or services, and other associated logic represent firmware, hardware, and/or software configured to control operations of the health monitoring system 2100. Such services may be implemented using a general purpose computer and specialized software (such as a server executing service software), a special purpose computing system and specialized software (such as a mobile device or network appliance executing service software), or other computing configurations. In addition, one or more functionalities of the health monitoring system 2100 disclosed herein may be generated by the processor 2802 and a user may interact with a Graphical User Interface (GUI) using one or more user-interface devices (e.g., the keyboard 2816, the display unit 2818, and the user devices 2804) with some of the data in use directly coming from online sources and data stores. The system set forth in FIG. 38 is but one possible example of a computer system that may employ or be configured in accordance with aspects of the present disclosure.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A machine-readable medium includes any mechanism for storing information in a form (e.g., software, processing application) readable by a machine (e.g., a computer). The machine-readable medium may include, but is not limited to, magnetic storage medium, optical storage medium; magneto-optical storage medium, read only memory (ROM); random access memory (RAM); erasable programmable memory (e.g., EPROM and EEPROM); flash memory; or other types of medium suitable for storing electronic instructions.

The description above includes example systems, methods, techniques, instruction sequences, and/or computer program products that embody techniques of the present disclosure. However, it is understood that the described disclosure may be practiced without these specific details.

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes.

While the present disclosure has been described with reference to various implementations, it will be understood that these implementations are illustrative and that the scope of the disclosure is not limited to them. Many variations, modifications, additions, and improvements are possible. More generally, implementations in accordance with the present disclosure have been described in the context of particular examples. Functionality may be separated or combined in blocks differently in various implementations of the disclosure or described with different terminology. These and other variations, modifications, additions, and improvements may fall within the scope of the disclosure as defined in the claims that follow.

What is claimed is:

1. A health monitoring device comprising:
a light source configured to emit photons into an optical waveguide, the photons internally reflected in the optical waveguide;
a compliant surface compressible against the optical waveguide during a scan of tissue, compression of the compliant surface against the optical waveguide scattering at least one of the photons back through the optical waveguide; and
an imaging array configured to collect the at least one scattered photon, forming an image representing a hardness of the tissue relative to surrounding tissue.

2. The health monitoring device of claim 1, wherein the tissue is breast tissue.

3. The health monitoring device of claim 1, further comprising:
a body having one or more contoured surfaces.

4. The health monitoring device of claim 3, wherein the body is sized to fit within a hand of a user and the one or more contoured surfaces mirror the shape of the hand.

5. The health monitoring device of claim 1, wherein the imaging array is a Charge-Coupled Device camera.

6. The health monitoring device of claim 1, wherein the compression of the compliant surface against the optical waveguide increases proportionally to the hardness of the tissue.

7. The health monitoring device of claim 1, wherein the compliant surface is pressed against a coupling material during the scan of the tissue.

8. The health monitoring device of claim 7, wherein the coupling material includes a guide pattern to guide the compliant surface during the scan of the tissue.

9. The health monitoring device of claim 1, wherein the diagnostic result includes a prompt to seek review by a medical professional for diagnosis.

10. One or more non-transitory tangible computer-readable storage media storing computer-executable instructions for performing a computer process on a computing system, the computer process comprising:
receiving an image sequence and corresponding location and orientation data captured by one or more sensors during a scan of tissue;
registering the image sequence based on the location and orientation data to form a map of the tissue; and
generating a diagnostic result based on the registered image sequence.

11. The one or more non-transitory tangible computer-readable storage media of claim 10, wherein the tissue is breast tissue.

12. The one or more non-transitory tangible computer-readable storage media of claim 10, wherein the diagnostic result includes an identification of potentially malignant cancer.

13. The one or more non-transitory tangible computer-readable storage media of claim 10, wherein the diagnostic result is generated based on a comparison of the registered image sequence to one or more previous image sequences.

14. The one or more non-transitory tangible computer-readable storage media of claim 13, wherein the diagnostic result is output for review on a user interface.

15. The one or more non-transitory tangible computer-readable storage media of claim 14, wherein the user interface is displayed on a graphical user interface of a health monitoring device.

16. A system for monitoring health comprising:
a housing having a body and a sensor head;
one or more sensors disposed in the sensor head and configured to generate a dynamic wave front signal during a scan of tissue; and
an imaging array disposed within the housing and configured to capture scan data from the dynamic wave front signal to form an image representing a hardness of the tissue relative to surrounding tissue.

17. The system of claim 16, further comprising:
at least one computing unit in communication with the imaging array, the at least one computing element configured to generate a diagnostic result indicating a potential presence of cancerous cells in the tissue, the diagnostic result generated based on the scan data.

18. The system for monitoring health of claim 17, wherein the diagnostic result is generated based on a comparison of the image to at least one previous image corresponding to a previous scan of the tissue.

19. The system for monitoring health of claim 16, wherein the one or more sensors includes at least one of an optical sensor or a static tactile sensor.

20. The system for monitoring health of claim 16, wherein the image is displayed on a user interface of a user device in communication with the imaging array.
